(19) United States
(12) Patent Application Publication
(10) Pub. No.: US 2017/0370908 A1
(43) Pub. Date: Dec. 28, 2017

(54) BRAIN IN VITRO MODELS, DEVICES, SYSTEMS, AND METHODS OF USE THEREOF

(71) Applicant: President and Fellows of Harvard College, Cambridge, MA (US)

(72) Inventors: Stephanie Dauth, Cambridge, MA (US); Josue A. Goss, Cambridge, MA (US); Thomas Grevesse, Somerville, MA (US); Ben Meir Maoz, Belmont, MA (US); Kevin Kit Parker, Cambridge, MA (US)

(73) Assignee: President and Fellows of Harvard College, Cambridge, MA (US)

(21) Appl. No.: 15/536,310

(22) PCT Filed: Dec. 17, 2015
(86) PCT No.: PCT/US15/66428

§ 371 Date: Jun. 15, 2017

Related U.S. Application Data
(60) Provisional application No. 62/093,235, filed on Dec. 17, 2014.

(51) Int. Cl. G01N 33/50; C12N 5/00; B01L 3/00

(52) U.S. Cl. CPC G01N 33/5058; B01L 3/5027; C12N 5/0062

(57) ABSTRACT

Exemplary embodiments provide in vitro brain models, such as in vitro models of a neurovascular unit or a functionally connected trineural pathway, and systems, devices and methods of use thereof. The present invention provides in vitro brain models, systems, devices and methods that mimic in vivo conditions to, for example, determine the effect of a test compound, such as a drug candidate or a toxin, on various biological responses, such as for example, cell viability, cell growth, migration, differentiation and maintenance of cell phenotype, metabolic activity, structural remodeling and tissue level pre-stress, a neural activity, such as an electrophysiological activity.

### RELATED APPLICATIONS

This application claims the benefit of co-pending, commonly assigned U.S. Provisional Patent Application No. 62/093,235, entitled “Neurovascular Unit Chip and Brain Chip Systems, Devices and Methods of Use Thereof”, filed on Dec. 17, 2014, the entire contents of which are incorporated herein by reference.

### BACKGROUND

The complexity of the human brain itself makes it a difficult organ to study, as it contains 86 billion neurons and 85 billion non-neuronal cells unevenly distributed and interconnected throughout various brain regions (See Herculano-Houzel, S., Frontiers In Human Neuroscience 3:31 (2009)). Indeed, he variability of cell morphology, size, functions, connections and gene expression is greater than that seen in any other organ (e.g., there may be approximately 700 types of neurons in the hippocampus alone) (See Stevens, C. F., Current Biology, CB 8(20):R708-710 (1998); and Masland, R. H., Current Biology, CB 14(13):R497-500 (2004)).

Given the complexities of the brain, there are a variety of challenges for designing an effective and accurate in vitro model of the brain. Currently, there is a lack of in vitro models that recapitulate the complexity of the brain. In general, the existing in vitro brain models utilize, at most, two cell region types in isolation and do not utilize more than two cell region types for reviewing the in vitro brain model as a multi-region unit.

In addition to the complexity of the neural connections between regions of the brain, brain and vascular cells form a complex functionally integrated signaling network that is known as the neurovascular unit (NVU). Signaling between the vascular and neural cells is a key physiological process regulating the interaction between peripheral circulation and brain activity.

Although some progress in the understanding of neurovascular coupling has been made, the complexity of the NVU also provides a variety of challenges for designing an effective and accurate in vitro model.

Accordingly, there is a need in the art for improved in vitro brain models, systems, devices and methods that mimic the complexity of the brain in vivo.

### SUMMARY

The present invention provides in vitro brain models, systems, devices and methods that mimic in vivo conditions to, for example, determine the effect of a test compound, such as a drug candidate or a toxin, on various biological responses, such as for example, cell viability, cell growth, migration, differentiation and maintenance of cell phenotype, metabolic activity, structural remodeling and tissue level pre-stress, a neural activity, such as an electrophysiological activity.

In accordance with one exemplary embodiment, an engineering neurovascular unit (NVU) is provided. As discussed herein, the NVU can include neuronal cells, astrocytes and endothelial cells for mimicking the NVU of the brain. The NVU includes an NVU structure including a hydrogel, e.g., hyaluronic acid (HA), scaffold configured to support neuron (e.g., cortical) and astrocyte growth and direct development of neuronal tissue. In some embodiments, induced pluripotent stem cells (iPS) can be used in the NVU structure. It should be understood that the NVU structure can include cells from a variety of sources, such as rat, human, or other species. The NVU also includes a first channel within the scaffold configured to support endothelial and pericyte cell growth. The NVU further includes a second channel within the scaffold and parallel to the first channel configured to support flow of angiogenic factors and the formation of gradients perpendicular to the first channel, and direct the formation of capillary-like channels (e.g., blood vessels) from the first channel in the direction of the second channel. In some embodiments, the HA scaffold can be formed by casting the HA gel such that the first and second channels are formed within the HA scaffold. The angiogenic factors within the second channel can trigger the formation of blood vessels from the first channel in the direction of the second channel.

In some embodiments, the NVU can include a channel input and a channel output connected to opposing sides of the first channel. In some embodiments, the NVU can include a channel input and a channel output connected to opposing sides of the second channel. The angiogenic factors can be at least one of VEGF, B-FGF, PMA, combinations thereof, or the like. The formation of capillary-like channels from the first channel can substantially mimic in vivo cell formation and structure.

In accordance with one exemplary embodiment, a device for in vitro evaluation of a neurovascular unit is provided. The device can include an NVU structure including a hydrogel, e.g., hyaluronic acid, scaffold configured to support neuron and astrocyte growth and direct development of neuronal tissue. The NVU structure includes a first channel within the scaffold configured to support endothelial and pericyte cell growth. The NVU structure includes a second channel within the scaffold and parallel to the first channel configured to support flow of angiogenic factors and the formation of gradients perpendicular to the first channel, and direct the formation of capillary-like channels from the first channel. The device can include a housing. The housing includes a chamber configured to house a corresponding NVU structure, an input channel connected to the corresponding chamber housing the NVU structure, and an output channel connected to the corresponding chamber housing the NVU structure.

In accordance with one exemplary embodiment, a system for in vitro evaluation of a neurovascular unit is provided that includes a plurality of NVU structures. At least one of the NVU structures includes a hydrogel, e.g., hyaluronic acid, scaffold configured to support neuron and astrocyte growth and direct development of neuronal tissue. At least one of the NVU structures includes a first channel within the scaffold configured to support endothelial and pericyte cell growth. At least one of the NVU structures includes a second channel within the scaffold and parallel to the first channel configured to support flow of angiogenic factors and the formation of gradients perpendicular to the first channel, and direct formation of capillary-like channels from the first channel. The system includes a housing including a plurality of chambers, a plurality of input channels, and a plurality of output channels. Each chamber can be configured to house a corresponding NVU structure. Each input channel can be connected to a corresponding chamber of the plurality of chambers. Each output channel can be connected to a corresponding chamber of the plurality of chambers.

In accordance with one exemplary embodiment, a method for identifying a compound which modulates an NVU function is provided. The method includes providing the NVU structure described herein and contacting the NVU with a test compound. The method includes determining the effect of the test compound on an NVU function in the presence and absence of the test compound. A modulation of the NVU function in the presence of the test compound as compared to the NVU function in the absence of the test compound indicates that the test compound modulates an NVU function, thereby identifying a compound that modulates the NVU function. In some embodiments, the NVU function can be selected from the group consisting of neuronal firing frequency, neuronal firing amplitude, capillary diameter, vasomotor tone, blood flow under shear stress, permeability of the endothelium, cell viability, excreted proteins, metabolic rate, use of nutrients, diffusion of O₂, combinations thereof, or the like. Determining the effect of the test compound on an NVU function in the presence and absence of the test compound can substantially mimic in vivo cell reactions.

The present invention further provides systems, devices and methods for forming an in vitro brain model that accurately and effectively models the multi-regional configuration of the brain.

In accordance with one exemplary embodiment, the present invention provides an engineered functionally connected trineural pathway. The engineered functionally connected trineural pathway includes a base layer, comprising a first independent plurality of cells comprising neurons and astrocytes obtained from a first brain region; a second independent plurality of cells comprising neurons and astrocytes obtained from a second brain region; a third independent plurality of cells comprising neurons and astrocytes obtained from a third brain region; a first independent plurality of axons functionally connecting the first plurality of cells to the second plurality of cells; a second independent plurality of axons functionally connecting the first plurality of cells to the third plurality of cells; and a third independent plurality of axons functionally connecting the second plurality of cells to the third plurality of cells. The first brain region may be the cortex; the second brain region may be the hippocampus; and the third brain region may be the amygdala.

In accordance with one exemplary embodiment, a device for in vitro evaluation of a neural function and/or a neural morphology is provided that includes an engineered functionally connected trineural pathway and a housing. The neural structure includes a base layer including a first independent plurality of cells comprising neurons and astrocytes obtained from a first brain region; a second independent plurality of cells comprising neurons and astrocytes obtained from a second brain region; a third independent plurality of cells comprising neurons and astrocytes obtained from a third brain region; a first independent plurality of axons functionally connecting the first plurality of cells to the second plurality of cells; a second independent plurality of axons functionally connecting the first plurality of cells to the third plurality of cells; and a third independent plurality of axons functionally connecting the second plurality of cells to the third plurality of cells. The housing includes a chamber configured to house a corresponding engineered functionally connected trineural pathway; an input channel connected to a corresponding chamber housing the engineered functionally connected trineural pathway; and an output channel connected to a corresponding chamber housing the engineered functionally connected trineural pathway.

In accordance with one exemplary embodiment, a system for in vitro evaluation of a neural function and/or a neural morphology is provided. The system includes a plurality of engineered functionally connected trineural pathways which include a base layer, comprising a first independent plurality of cells comprising neurons and astrocytes obtained from a first brain region; a second independent plurality of cells comprising neurons and astrocytes obtained from a second brain region; a third independent plurality of cells comprising neurons and astrocytes obtained from a third brain region; a first independent plurality of axons functionally connecting the first plurality of cells to the second plurality of cells; a second independent plurality of axons functionally connecting the first plurality of cells to the third plurality of cells; and a third independent plurality of axons functionally connecting the second plurality of cells to the third plurality of cells. The system includes a housing including a plurality of chambers, a plurality of input channels, and a plurality of output channels. Each chamber can be configured to house a corresponding neural structure. Each input channel can be connected to a corresponding chamber of the plurality of chambers. Each output channel can be connected to a corresponding chamber of the plurality of chambers.

In accordance with one exemplary embodiment, a method for identifying a compound which modulates a neural function and/or a neural morphology is provided. The method includes providing a device as described herein and contacting the engineered functionally connected trineural pathway of the device with a test compound. The method includes determining the effect of the test compound on a neural function and/or a neural morphology in the presence and absence of the test compound. A modulation of the neural function and/or neural morphology in the presence of the test compound and compared to the neural function and/or neural morphology in the absence of the test compound indicates that the test compound modulates a neural function, thereby identifying a compound that modulates a neural function and/or neural morphology. In some embodiments, the neural function can be an electrical function.

In accordance with one exemplary embodiment, a method of forming an engineered functionally connected trineural pathway is provided. The method includes providing a base layer, said base layer comprising three independent regions; depositing a pattern of extracellular matrix protein onto the base layer, wherein the pattern comprises a first plurality of lines connecting the first independent region with the second independent region, a second plurality of lines connecting the first independent region with the third independent region, and a third plurality of lines connecting the second independent region with the third independent region, placing a patterned mask onto the base layer, wherein the patterned mark comprises openings corresponding to the first, second and third regions, seeding neural cells and astrocytes obtained from a first area of the brain onto the first region; seeding neural cells and astrocytes obtained from a second area of the brain onto the second region; and seeding neural cells and astrocytes obtained from a third area of the brain onto the third region; culturing the cells for a time sufficient for the cells in each region to adhere to the base layer; removing the patterned mask and further culturing the cells for a time sufficient for the cells from the first and second areas of the brain to functionally connect, for the cells from the first and third areas of the brain to functionally connect, and for the cells from the second and third areas of the brain to functionally connect, thereby generating the engineered functionally connected trineural pathway.

The base layer may be, e.g., a glass coverslip or a petri dish, and may further comprise a multi-electrode array, e.g., for electrophysiological assessments.

The extracellular matrix protein may be one or more of fibronectin, laminin, bevican, aggrecan, tenascin-R, and combinations thereof.

The neural cells and astrocytes may be independently obtained from a cortex region, a hippocampus region, and an amygdala region of the brain.

The methods may further include depositing a cell adherent substance, e.g., poly-L-lysine, on one or more of the three regions on the base layer.

### BRIEF DESCRIPTION OF THE DRAWINGS

The foregoing and other objects, aspects, features, and advantages of exemplary embodiments will become more apparent and may be better understood by referring to the following description taken in conjunction with the accompanying drawings, in which:

FIG. 1 illustrates a block diagram of an exemplary engineering strategy for constructing an NVU on a chip.

FIGS. 2A-D illustrate schematics of an exemplary in vitro NVU, including a top view with platform dimensions (FIG. 2A), an inset of channels created in the system (FIG. 2B), and astrocytes cultured in a three-dimensional gel and endothelial cells in the channels (FIGS. 2C and 2D).

FIG. 3 illustrates a fluorescent dye flowed through a channel of an exemplary NVU system created in a hydrogel.

FIG. 4 illustrates a flow velocity profile in channels for different flow rates by measuring velocity of fluorescent beads perfused in channels of an exemplary NVU system.

FIG. 5 illustrates shear stress values for an exemplary NVU system obtained from an experimental flow profile compared with theoretical values calculated with Poiseuille's Law.

FIG. 6 illustrates an assay used to measure stress applied by pericytes treated with the vasoactive agent, endothelin.

FIG. 7 illustrates stress measurement applied by pericytes treated with vasoactive agent endothelin.

FIGS. 8A-C illustrate measurements of vasomotor tone and neuronal firing in an NVC (FIG. 8A), a correlation matrix between the vasomotor tone (VT) and the neuronal firing (NF) (FIG. 8B), and the result of system performance as a function of toxicant concentration (FIG. 8C).

FIG. 9 illustrates a perspective view of an exemplary NVU system.

FIG. 10 illustrates a top view of an exemplary NVU system.

FIG. 11 illustrates a top view of an exemplary NVU system.

FIG. 12 illustrates a side view of an exemplary NVU system.

FIG. 13 illustrates a cross-sectional view of an exemplary NVU system.

FIG. 14 illustrates a cross-sectional view of an exemplary NVU system.

FIG. 15 illustrates an in vivo cortex brain section.

FIG. 16 illustrates in vitro cortical neurons.

FIG. 17 illustrates a corticolimbic system.

FIG. 18 illustrates an exemplary trineural pathway in vitro.

FIG. 19 illustrates a flowchart of a process of forming an exemplary in vitro trineural pathway.

FIG. 20 illustrates functional connection of regions in an exemplary trineural pathway comprising a first region comprising cells obtained from the prefrontal cortex (pfCx), a second region comprising cells obtained from the hippocampus, and a third region comprising cells obtained from the amygdala.

FIG. 21 illustrates a magnified view of the exemplary functionally connected trineural pathway in FIG. 20.

FIG. 22 illustrates an image of a polydimethylsiloxane (PDMS) mask on a glass coverslip in a 12-well plate.

FIG. 23 illustrates PDMS masks on a multi-electrode array (MEA).

FIG. 24 illustrates multi-electrode array data functional connection (electrical connection) of a trineural pathway model.

FIG. 25 illustrates a cross-correlation of electrical activity of three different brain regions of a trineural pathway in vitro model.

FIGS. 26A-J illustrate a distribution of brain extracellular matrix proteins in vivo.

FIG. 27 illustrates a diagrammatic representation of microcontact printed Aggrecan and Laminin on a coverslip.

FIG. 28 illustrates ECM protein expression in different regions of a brain.

FIG. 29 illustrates ECM protein expression in vitro relative to in vivo.

FIG. 30 illustrates protein expression and electrical activity analysis of distinct brain regions in vitro.

FIG. 31 illustrates a comparison of protein expression in vivo and in vitro neurons.

FIG. 32 illustrates immunostaining of a microenvironment.

FIG. 33 illustrates a number of Aggrecan positive cells.

FIG. 34 illustrates mass spectrometry for Aggrecan.

FIG. 35 illustrates mass spectrometry for Brevican.

FIG. 36 illustrates mass spectrometry for Tenascin-R.

### DETAILED DESCRIPTION

Although some in vitro models of the brain have been generated, they do not recapitulate the complexity of the brain in vivo. For example, although some progress in the understanding of neurovascular coupling has been made, disruption caused by toxicants remains poorly understood. In vivo, toxicants reaching the neurons first have to pass the neurovascular unit, as opposed to in vitro models where toxicants are added directly to the cells.

Accordingly, in one aspect, the present invention solves these problems by providing systems, devices and methods for measuring in vitro responses of a neurovascular unit. In particular, the exemplary systems accurately recreate the NVU in vitro and allow for efficient and accurate monitoring of the neurovascular functioning in vitro. The exemplary systems are versatile and can be used for different applications (e.g., basic scientific questions related to the neurovascular coupling, drug screening, toxicant analysis, and the like).

In vivo, all exogenous chemicals reaching neurons pass through the neurovascular unit. Current in vitro models neglect this fact, and chemicals are added directly to the neuronal cultures, not through the neurovascular unit. The exemplary platform disclosed herein mimics in vivo conditions; thereby the chemicals are added to in vivo-like blood vessels and pass through the neurovascular unit before reaching the neurons. Moreover, by optically monitoring the vasomotor tone (VT), the exemplary systems provide improved means for analyzing the effects of test compounds and/or toxins on neuronal functionality. Therefore, the platform provides a novel tool that incorporates the brain microenvironment, three-dimensional, capillary-like micro channels (<15 μm) and each of the cell types forming the neurovascular unit (including the blood-brain-barrier).

In particular, the exemplary systems provide an accurate testing configuration of an in vitro neurovascular unit. The exemplary system accurately recreates a network of interconnected micro channels mimicking a network of brain blood capillaries and their surrounding tissue. The use of an engineered scaffold and multiple cell types (e.g., neurons, astrocytes, pericytes and endothelial cells) permits the recapitulation of a neurovascular unit at the capillaries in an environment mimicking the native environment. The system recapitulates the contraction/dilation of blood capillaries in response to the neuronal electrical activity. The system can be perfused with any desired fluid and can be used to test the effects of toxicants and drugs on brain neuronal networks in a biological relevant environment. The system can also be used to answer fundamental questions on neurovascular coupling whose study has been impaired by a lack of adapted tools.

A variety of applications or implementations of the system are possible. With respect to toxicant testing, the in vitro neurovascular unit can be used to test the passage of toxicants through the human blood-brain-barrier and tests the effects of toxicants on three-dimensional neurons and glial cells tissues. With respect to drug testing, the in vitro neurovascular unit can be used to assay drug candidates during drug screening or drug validation tests or as a direct indicator of drug efficacy and toxicity. With respect to fundamental research, the in vitro neurovascular unit can be used to study fundamental questions and phenomena underlying the coupling of brain blood flow and brain electrical activity in health and diseases.

The composition of the exemplary systems can include a cell scaffold, cells, a microfluidic chamber, combinations thereof, or the like. The scaffold can be synthetic or natural depending on the application. The scaffold can be built through the cross-linking of hydrogel pre-solutions, such as collagen, gelatin, agarose, hyaluronan, combinations thereof, or the like. Cell scaffolds suitable for the NVU are biocompatible, mechanically-stable (typically performed by cross-linking) throughout biologically relevant temperatures (e.g., approximately 21° C.-40° C.), and amenable to cell adhesion, either directly or by functionalization.

Suitable cell types which can be used to build the in vitro neurovascular unit include endothelial cells, pericytes, astrocytes and neurons. In some embodiments, other types of cells can be added to these cell types used depending on the situation and specific research question, such as red and white blood cells, oligodendrocytes, microglia, combinations thereof, or the like. The cells used in the in vitro neurovascular unit can come from virtually any sources. For example, cells derived from human induced pluripotent stem cell lines can be used to create an in vitro neurovascular unit with, e.g., patient specific features. Other suitable cell sources include other stem cell-derived sources and primary mammalian cells, such as neonatal rodent brain cells.

A microfluidic chamber can be used in the system. All the pieces of the microfluidic chamber can be compatible with biological materials and can be designed and constructed with biologically-inert materials. In some embodiments, one or more portions of the microfluidic chamber can be fabricated from polycarbonate, acrylic (PMMA), cyclic olefin polymer (COP), cyclic olefin co-polymer (COC), polyetherimide (PEI), combinations thereof, or the like. Categorically, the microfluidic components can be manufactured from thermoplastics, thermoset polymers, silicons (e.g., glass, quartz, or the like), metals (e.g., 316 Stainless, titanium, or the like), combinations thereof, or the like.

In some embodiments, the exemplary systems are developed to mimic the fetal developmental state of the neurovascular unit, since the nascent central nervous system (CNS) is most susceptible to toxicants. In particular, toxicant exposure for the fetal brain can occur through inhalation exposure (e.g., PAH, nicotine, toluene, or the like), oral exposure (e.g., alcohol, food contaminants, pesticizers (DDT, DDE), or the like), transfer (e.g., lipophilic chemicals to offspring by breast feeding, from mother to fetus and/or to amniotic fluid, or the like). Thus, toxicants inhaled or ingested by the mother can find their way to the fetus through the blood and potentially affect the developing CNS. It should be understood, however, that the exemplary systems described herein can be used to mimic the fetal and/or mature or adult neurovascular unit.

The exemplary systems provide an in vitro NVU model that recapitulates the important features of the in vivo NVU. The systems enable assessment of, for example, drug candidates or toxicant, effects on neuronal network functionality by optically observing the VT. In particular, the system includes a three-dimensional design of a tissue engineered platform that recapitulates the cellular architecture of the NVU. The high throughput system is a biologically relevant system that can be used in the pharmaceutical industry and correlates to the in vivo NVU, thereby being of clinical relevance.

The systems allows for simulation of features of interest during use and allows for adjustment of parameters in the design of the system, such as flow profile, diffusion rate, combinations thereof, or the like.

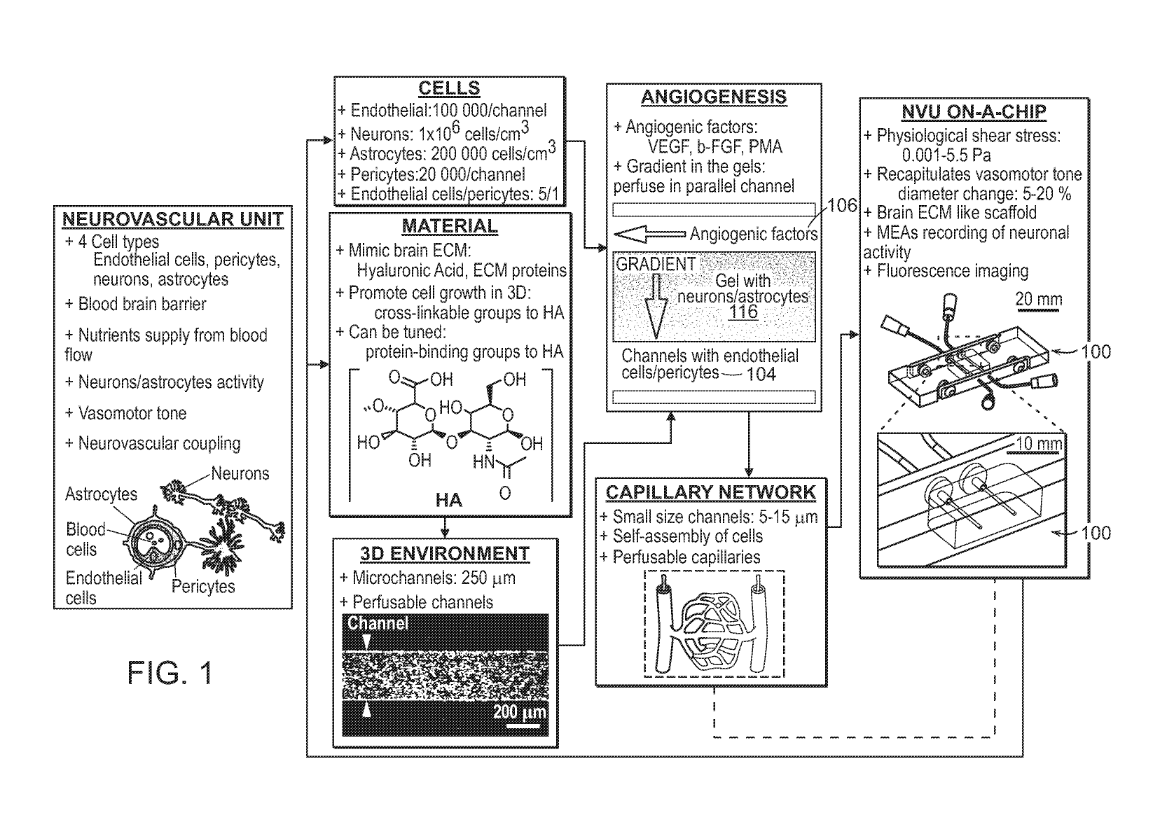

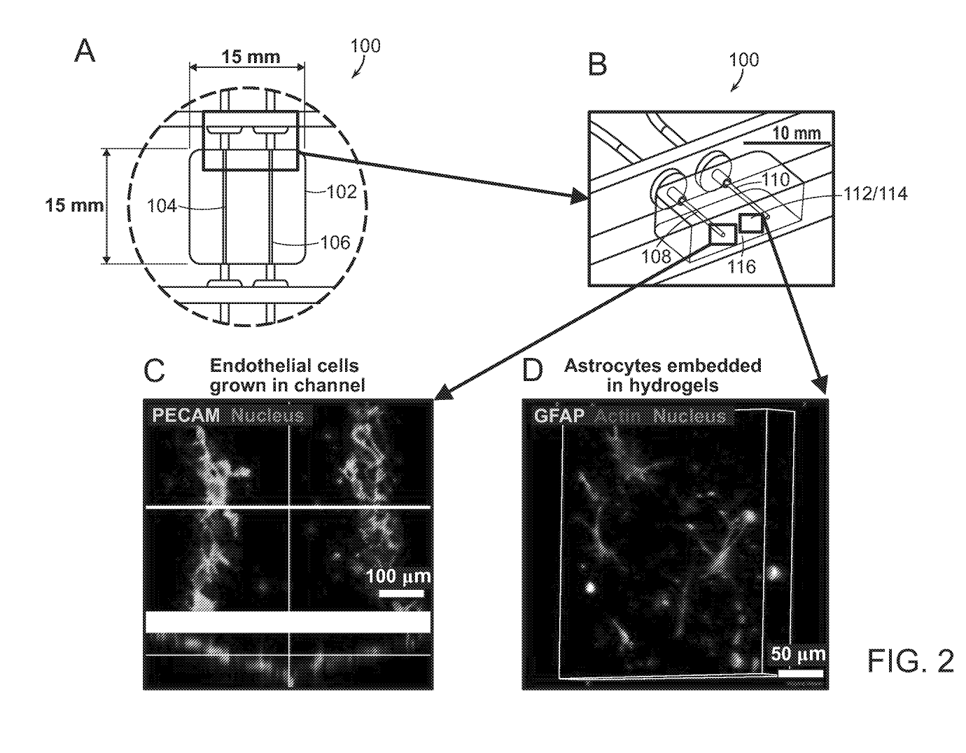

FIG. 1 illustrates a block diagram of an exemplary engineering strategy for constructing an NVU on a chip, hereinafter system 100. The exemplary microfluidic chip incorporates the engineered NVU and allows in vitro measurement and correlation of VT to neuronal firing activity. FIGS. 2A-D illustrate schematics of an exemplary in vitro NVU system 100. The chip was designed using SolidWorks®. NVU system 100 is characterized by unique features and environment. The NVU function relies on the appropriate cross-talk of the four main cell types: neurons, astrocytes, pericytes and endothelial cells. This allows the NVU to provide neurons with adequate trophic support while preventing most blood-borne pathogens to pass from blood to the neurons.

These specific features can be used as design specifications for building the in vitro NVU system 100. The system 100 includes a body 102 that defines a substantially rectangular or square configuration with chamfered edges. The body 102 can include two or more channels 104, 106 formed in the body 102 and extending between two opposing ends of the body 102. The channels 104, 106 can extend substantially parallel to each other.

For the scaffold (e.g., body 102) of the platform, hyaluronic acid (HA) was used as this is the main structural biopolymer in the brain, and a material that can be chemically modified and functionalizes with any desired protein. In addition, promotion of cell growth in the three-dimensional platform can be achieved by cross-linkable groups with the HA 116. In some embodiments, tuning of protein-binding groups to the HA 116 can be achieved. For example, HA was chemically modified to graft methacrylate groups to HA chains (see Bencherif, S. A., et al., Influence of the degree of methacrylation on hyaluronic acid hydrogels properties, Biomaterials, 2008, 29(12): p. 1739-49) and was further modified with N′-ethylcarbodiimide hydrochloride and N-hydroxysuccinimide in order to graft protein-binding groups to HA. This strategy provides the ability to graft any desired protein from the ECM to the HA gel and thereby provides the ability to control the ECM composition of the scaffolds.

The HA gel was cast around wires (e.g., wires having a diameter between 150 microns and 1 mm) to form the channels 104, 106. After casting, the wires were removed to leave behind the channels 104, 106. In particular, micro channels were created using the following technique: micro wires (250 μm) were placed in a frame, an HA solution was cast around the wires and exposed to UV light to cross-link the HA and form the gel. The micro wires were removed from the HA gel, thereby exposing the formed micro channels.

Angiogenic factors (e.g., VEGF, b-FGF, PMA, or the like), were introduced into a channel parallel to the cell-populated channel to create a gradients in the gel that triggered angiogenesis from the cell channel and formation of capillaries with patent lumen of 5 to 15 μm. In some embodiments, the channels 104, 106 can be substantially symmetrical such that both channels 104, 106 can be seeded with cells or perfused with angiogenic factors. It should be understood that a channel perfused with the angiogenic factors is not seeded by the cells. In particular, the angiogenic factors triggered and promoted the growth of blood vessels from the channel populated with cells to the channel with the angiogenic factors. For example, the blood vessels or capillaries grew from the channel populated with endothelial cells 108 and pericytes 110 in the direction of the channel with the angiogenic factors.

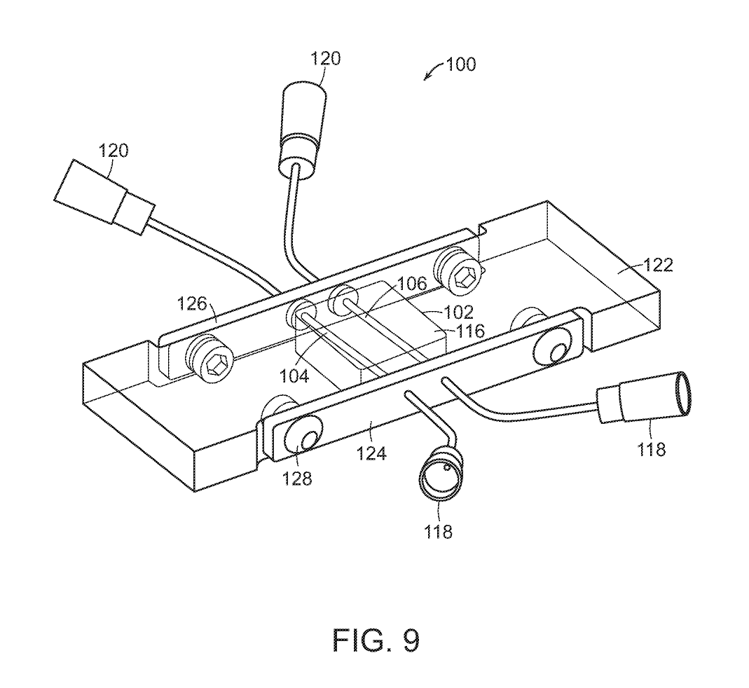

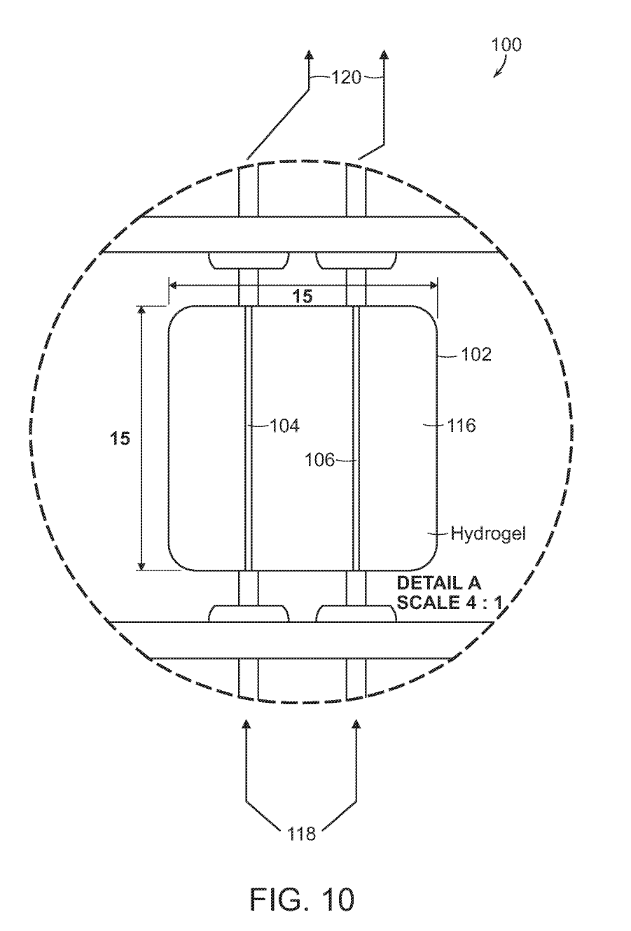

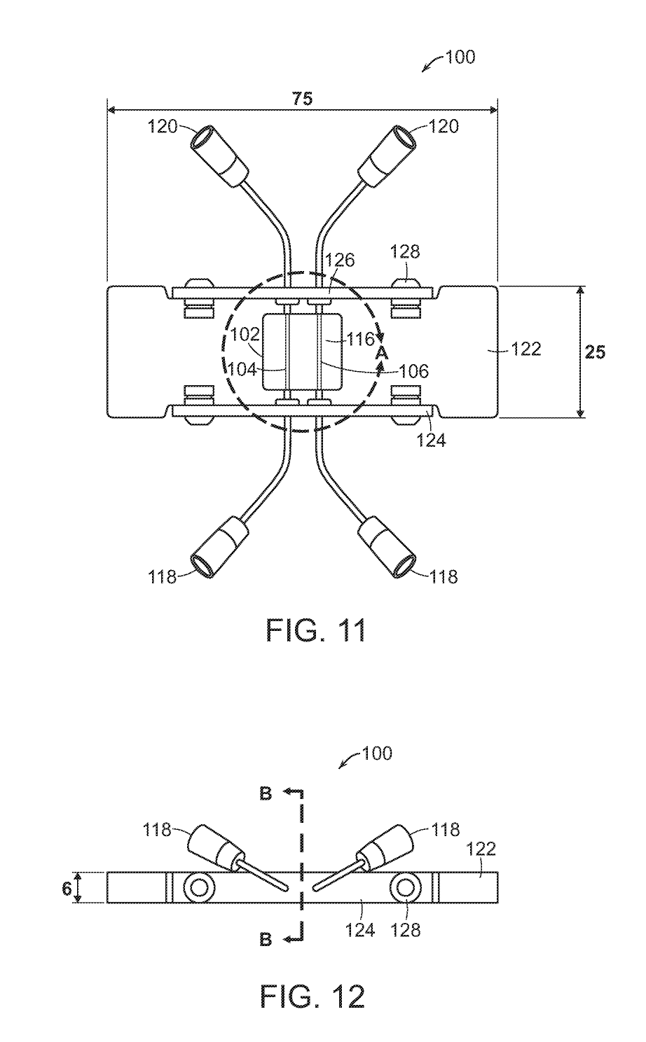

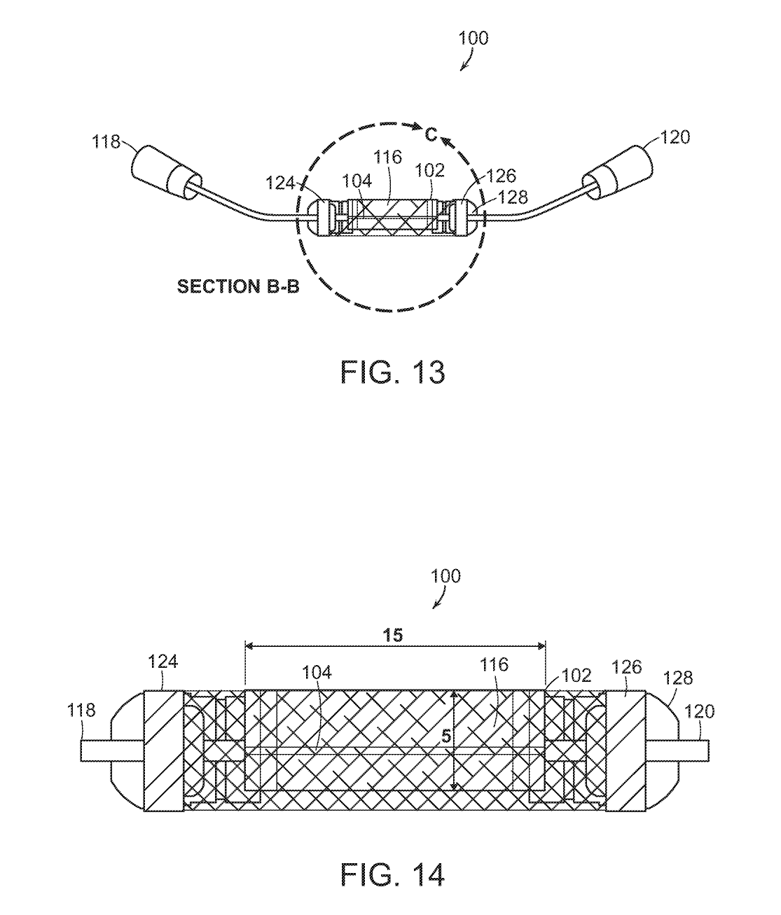

FIGS. 9-14 illustrate perspective, top, side and cross-sectional views of the exemplary NVU system 100 discussed above. The system includes the HA 116 with channels 104, 106 formed therein. Each channel 104, 106 can include an inlet 118 and an outlet 120 for introduction of, e.g., toxicants, into the respective channels 104, 106. In some embodiments, the system 100 can include a platform 122 configured to house the HA 116 therein. The system 100 can include opposing side panels 124, 126 secured with fasteners 128 to the platform 122 to maintain a position of the inlets 118 and outlets 120. The NVU system 100 can be used to assess neurovascular coupling under physiological and pathological conditions. Astrocytes and neurons can be embedded in the HA gels to form the “brain parenchyma”. Endothelial cells and pericytes can be seeded in one of the channels 104, 106 (one or the other) and allowed to cover the surface of the channel 104, 106. Next, in the other channel 104, 106, a mix of angiogenic factors (aforementioned) can be introduced or flowed. The angiogenic factors diffuse from the channel 104, 106 to the gels which creates a gradient in the gels. The angiogenic factor gradient triggers the formation of capillaries from the channel 104, 106 populated with endothelial cells and pericytes. Signaling between the four cell types allows the formation and maintenance of the capillaries that mimic the neurovascular unit.

Upon casting of the HA hydrogels, formation of the NVU was initiated by flowing pericytes and endothelial cells in channels created in the engineered, astrocyte and neuron-laden HA hydrogels. To trigger angiogenesis, medium containing angiogenic factors (e.g. VEGF-A, basic fibroblast growth factor, Phorbol-12-myristate-13-acetate, or the like) were flowed in a channel parallel to the main channel (see Tourovskaia, A., et al., Tissue-engineered microenvironment systems for modeling human vasculature, Exp. Biol. Med. (Maywood), 2014, 239(9): p. 1264-71). These factors diffused into the gels and created gradients perpendicular to the channels populated with endothelial cells, promoting the formation of capillary-like channels from the main channel. In particular, the angiogenic factors triggered the formation of the blood vessels between the channels.

The functionality of the NVU can then be tested. In particular, the cell architecture and the presence of a basement membrane (e.g., fibronectin, laminin, collagen IV, or the like) can be tested with immunostaining, the flow through the channels with fluorescent tracers, the nutrient supply with fluorescent analog of glucose, the neuronal firing rate with micro-electrode arrays (MEAs) and the VT with optical imaging of the diameter of the capillaries. Physiological shear stress of the system 100 can be tested between 0.001-5.5 Pa. Recapitulation of the vasomotor tone diameter can be changed between 5-25%. The MEAs can record the neuronal activity. Fluorescence imaging can be using for imaging of the capillaries.

The developmental state of the NVU was evaluated using quantitative real time-polymerase chain reaction (qRT-PCR) gene expression profiling. In particular, a physiologically relevant micro environment was created to support the variety of cell populations present in the NVU and assess the ability of the micro environment to recapitulate the appropriate developmental phenotype.

Fluorescence imaging was used to assess the diffusion rates of dextrans from the channels to the gels, the neuronal network viability and glucose consumption in the tissue surrounding the in vitro capillaries network. Testing was performed to determine if the angiogenesis-guided capillaries network recapitulates the permeability of the BBB, and can provide energy and nutrients from the flow to the neuronal networks.

MEAs and/or Ca²+ dyes were used to measure the firing activity of neuronal networks in the in vitro NVU, optically determined the change in capillary diameter and correlated it to the neuronal firing activity. Such testing measured and characterized the firing activity of neuronal networks and developed a metric for assessing the state of the NVU by determining a relationship between the VT and neuronal firing activity.

It was hypothesized that recapitulation in vitro of the features of the NVU as well as development of an analytic metric correlating the VT with the neuronal firing activity to assess the state of the NVU, the NVC and neuronal firing activity would be possible with the exemplary systems. VT is the dynamic change of the blood vessel diameter, a central process in NVC. Pericytes are responsible for dilation and contraction of capillaries in response to paracrine signaling, and thus regulate VT. On one side, pericytes are connected to endothelial cells, on the other side, pericytes interact with astrocytes. The VT that is controlled by the relaxation/contraction of pericytes is therefore dependent on the functional state of neurons, astrocytes, endothelial cells and pericytes. Any dysfunction related to the NVU generally affects the VT.

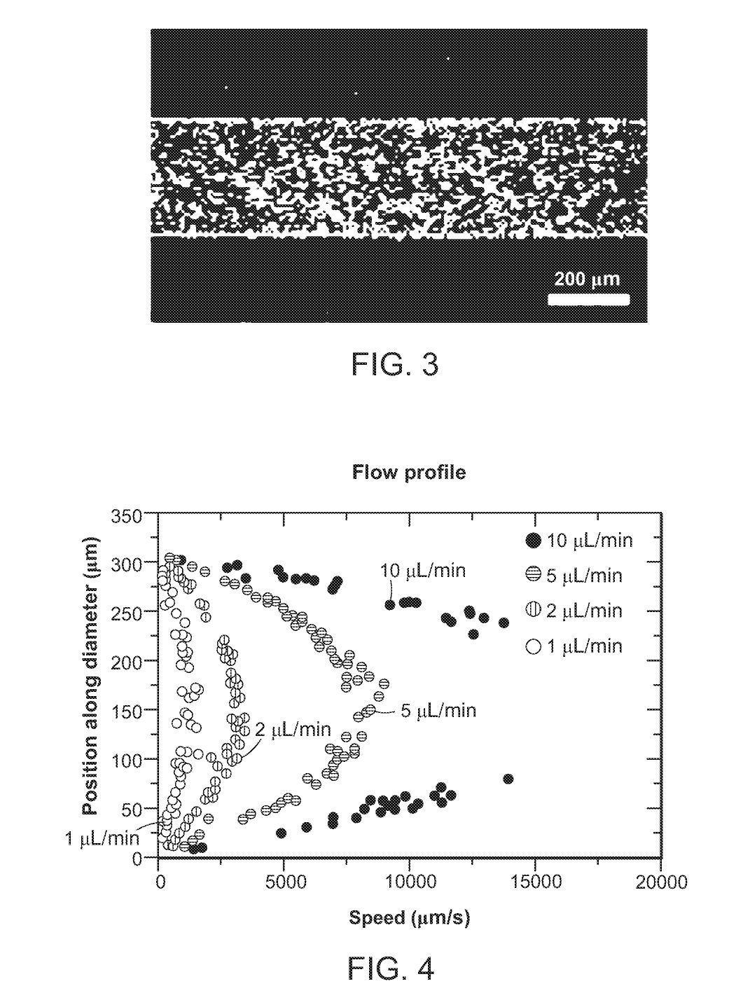

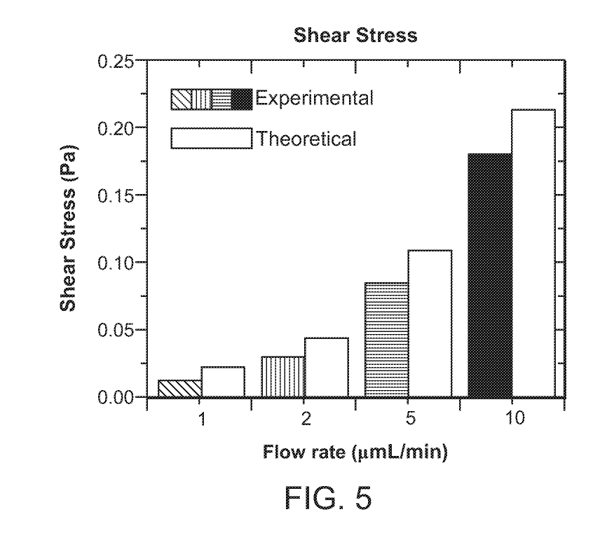

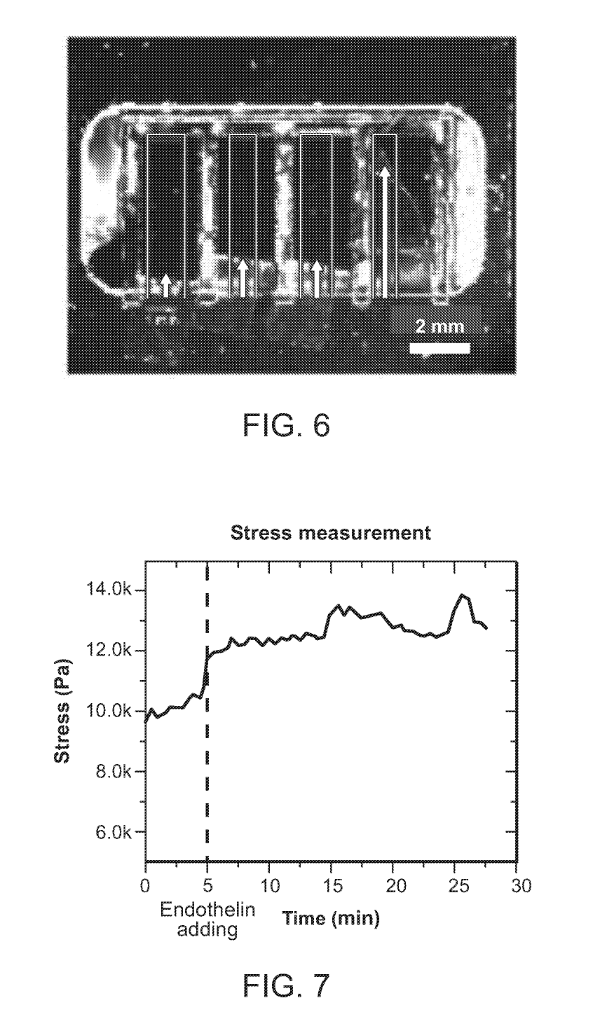

To assess the contraction response of pericytes to vasoactive agents, muscular thin films (MTFs) (see Feinberg, A. W., et al., Muscular thin films for building actuators and powering devices, Science, 2007, 317(5843): p. 1366-70) were used and it was observed that endothelin induces pericyte contraction, as shown in FIGS. 3-7.

In order to test if the hiPSC can replicate this developmental stage, qRT-PCR was performed and focused on genes that are expressed in the developing NVU, such as transforming growth factor one and three and α5β8 integrin in astrocytes (see McCarty, J. H., Integrin-mediated regulation of neurovascular development, physiology and disease, Cell Adh. Migr., 2009, 3(2): p. 211-5), Foxc transcription factor in pericytes (see Siegenthaler, J. A., et al., Foxc1 is required by pericytes during fetal brain angiogenesis, Biol. Open, 2013, 2(7): p. 647-59) and expression of VEGF receptor two in endothelial cells (see Bautch, V. L., et al., Neurovascular development: The beginning of a beautiful friendship, Cell Adh. Migr., 2009, 3(2): p. 199-204). Next, a medium with fluorescent dextrans was flowed through the main channels to assess the attainment of the capillaries formed by angiogenesis. The in vitro NVU recapitulates the important features of the NVU: spatial organization of the four cell types, BBB (blood brain barrier) formation and permeability, neurons and astrocytes functionality.

A series of immunostaining experiments was performed, staining the four different cell types and specific markers thereof (see Hawkins, B. T., et al., The blood-brain barrier/neurovascular unit in health and disease, Pharmacol. Rev., 2005, 57(2): p. 173-85) (e.g. aquaporins, endothelial tight junctions, gap junctions, proteins of the basal lamina: laminin, fibronectin, collagen IV, or the like) to assess whether their spatial organization mimics the in vivo situation. Next, the engineered capillaries were tested to determine if the capillaries recapitulate the characteristics of brain capillaries: (i) an efficient BBB, (ii) the ability to transport nutrients from the channels to the neuronal networks, and (iii) contraction/dilation of the channels in response to vasoactive agents.

The permeability of the BBB was tested by flowing fluorescent dextrans through the engineered microvascular channels both with and without cells. The dextrans diffuse in the gel and the diffusion coefficient can be calculated by measuring the fluorescence intensity in the gel surrounding the channels as a function of time. To assess the ability of the capillary to sustain the surrounding neuronal networks, the in vitro NVU was cultured in serum free, glucose free medium. The only source of nutrients came from a blood substitute delivered through the engineered microvasculature. Next, a fluorescent analog of 2-deoxy-D-glucose was flowed into the channels and imaging of the metabolism of glucose was made. This allowed testing of the ability of the created capillaries to provide nutrients and energy to surrounding neuronal networks.

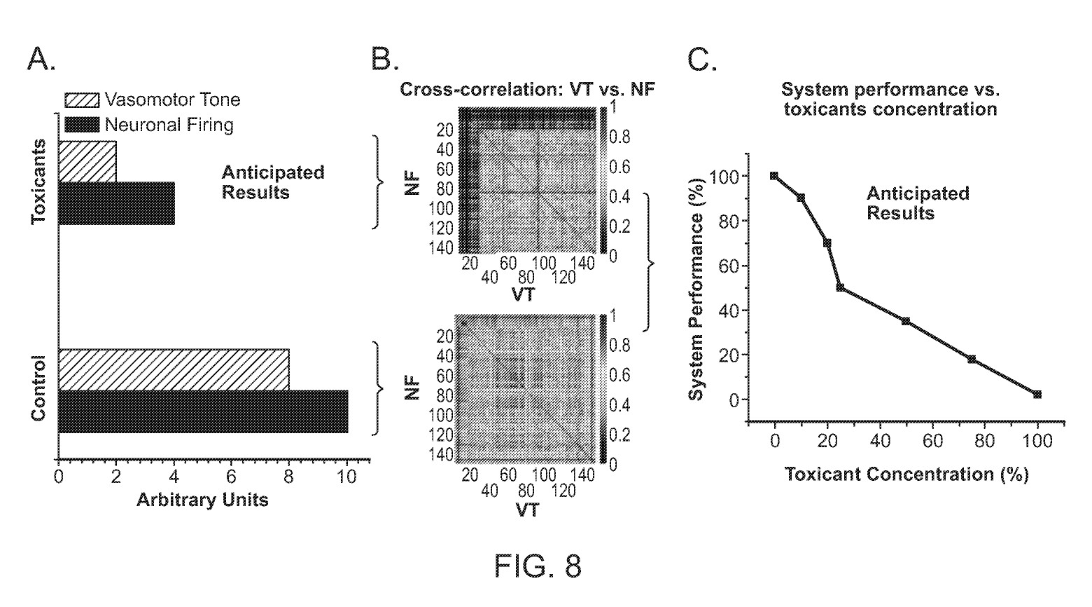

Measurements of neuronal activity were made using flexible MEAs, in addition to Ca²+ imaging using Ca²+ sensitive dyes (Fluo-4). Accordingly, the frequency and amplitude of neuronal firing activity was measured. Once the neuronal firing activity was recorded, stimulation of these networks was performed and both the neuronal firing activity and optically assessment of the diameter change of the capillaries (VT) was recorded. The time course of the VT allows derivation of the frequency and amplitude of the capillary diameter change. These parameters were correlated with the frequency and amplitude observed for neuronal firing activity. The correlation was used as a metric for assessing the state of NVC, as shown in FIGS. 8A-C. Because many parameters regulate the NVC (e.g., different cell types, neuronal firing activity, cellular dysfunction, environmental factors, or the like) any change in the correlation indicates a change in one of these parameters.

The exemplary in vitro NVU system can be configured to act independently of these parameters and, therefore, can relate the parameters to specific changes in the correlation. Therefore, the system can be used to assess the NVC state in response to toxicants in an environment mimicking the natural NVU. To assess the global change of state of the NVU (neuronal firing activity, VT, NVC, substances absorbed/released) due to the action of different toxicants, a quality index (e.g., Sheehy index) was used which provided an overall measure for the different NVU states (e.g. healthy vs after toxicant exposure) (See Sheehy, S. P., et al., Quality metrics for stem cell-derived cardiac myocytes, Stem Cell Reports, 2014, 2(3): p. 282-94).

Testing of the in vitro NVU system was also performed by evaluating the effects of toxicants on NVC and neuronal activity. The micro channels were perfused with known toxicants (e.g., alcohol, toluene and PAHs), an assessment of their effect on the NVC with the quality index score was determined, and measurement of the changes in the chemical concentration and composition of the solution entering and exiting the micro channels was made. The experimentation showed that the system can mimic neuronal dysfunction due to known toxicants, can be used to study effect of toxicants on NVC and assess which chemicals get absorbed, degraded or released after interacting with the cells of the NVU.

To assess the effect of alcohol, toluene and PAHs on NVC, the system was perfused with medium containing doses of alcohol similar to these observed in alcoholic patients' blood (2-10 mmol/L) (See Frezza, M., et al., High blood alcohol levels in women. The role of decreased gastric alcohol dehydrogenase activity and first-pass metabolism, N. Engl. J. Med., 1990, 322(2): p. 95-9), doses of toluene correlating to 100-1000 ppm (See Huang, J., et al., Dose dependent effects of chronic exposure to toluene on neuronal and glial cell marker proteins in the central nervous system of rats, Br. J. Ind. Med., 1992, 49(4): p. 282-6) and doses of 3-30 μm PAH (See Tang, Y., et al., Neurotoxicity of polycyclic aromatic hydrocarbons and simple chemical mixtures, J. Toxicol. Environ. Health A., 2003, 66(10): p. 919-40). Two capillary networks were built in the same gel. One was exposed to the toxicant and the other one to regular medium as the control experiment. Measurements of neuronal firing activity (MEAs) and channel diameter (microscopy) were performed frequently every day. For each recording, the amplitude and frequency of the diameter change and neuronal firing activity were calculated and correlated. In addition, mass spectrometry was used to assess changes in chemicals entering and exiting the system. This indicated which chemicals were absorbed, degraded or released by the system. For each day, the quality index was calculated to compare the healthy and intoxicated channels. This strategy allowed assessing the effect of environmental toxicant on the neuronal firing activity and the NVC. Moreover, the strategy used provided the opportunity to determine if dysfunctions in neuronal networks were responsible for later NVC dysfunction or if toxicants first impair the NVC that results in secondary neuronal dysfunction.

The anticipated results for toxicant exposure of the NVU were as follows. For the toxicant of alcohol, the neuronal firing activity and the vasomotor tone are expected to include periods of abnormally high and abnormally low states corresponding to the alcohol dosing and withdrawal. For the toxicant of toluene, the neuronal firing activity and the vasomotor tone are expected to include reduction due to neuronal loss and gliosis. For the toxicant of PAHs, the neuronal firing activity is expected to include reduction due to increase in oxidative stress, and the vasomotor tone is expected to include reduction due to increase in oxidative stress and neuronal loss.

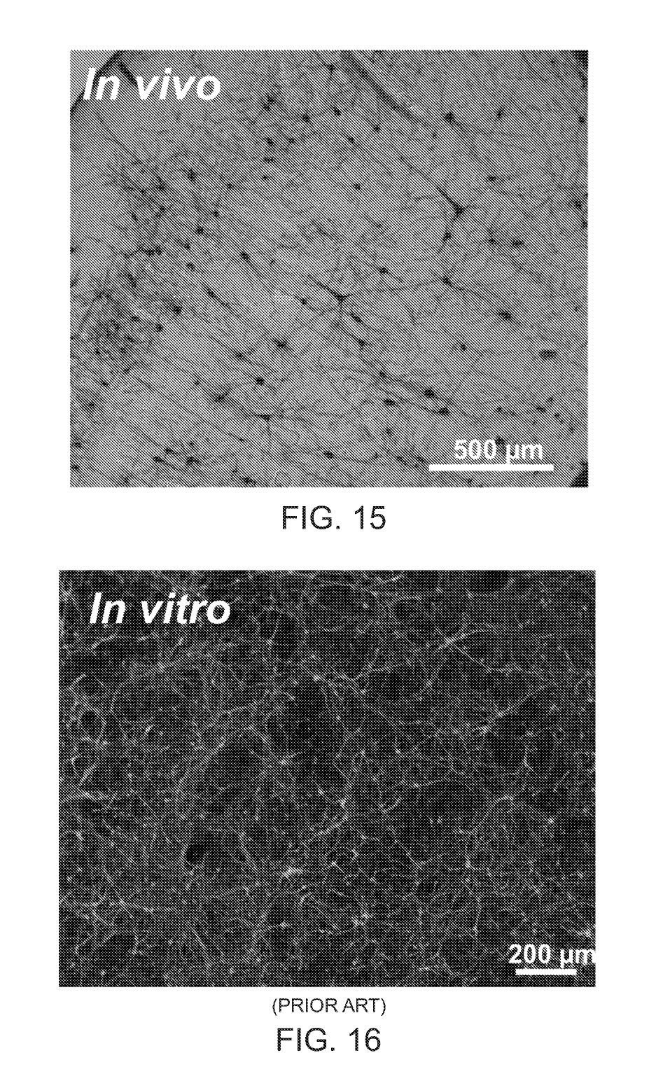

In another aspect of the present invention, a relevant in vitro model of a trineural pathway is provided which mimics the complex interconnection of brain regions in vivo As discussed above, current in vitro brain models implement cells from only two brain regions and do not reflect a multi-regional model of the brain. FIG. 15 illustrates an in vivo brain section cortex and FIG. 16 illustrates traditional in vitro cortical neurons used in the industry. Thus, a need exists in the industry for brain in vitro models which recapitulate in vivo characteristics (e.g., micro-environment, different brain regions, axonal connections, or the like). Therefore, in accordance with some embodiments of the present disclosure, exemplary in vitro brain models including cells from three brain regions are provided. The exemplary systems described herein evaluate in vivo features and recapitulate them in vitro. The exemplary engineered functionally connected, e.g., electrically connected, trineural pathways of the invention are versatile and can be used for different applications (e.g., basic scientific questions related to the cell coupling, drug screening, toxicant analysis, and the like).

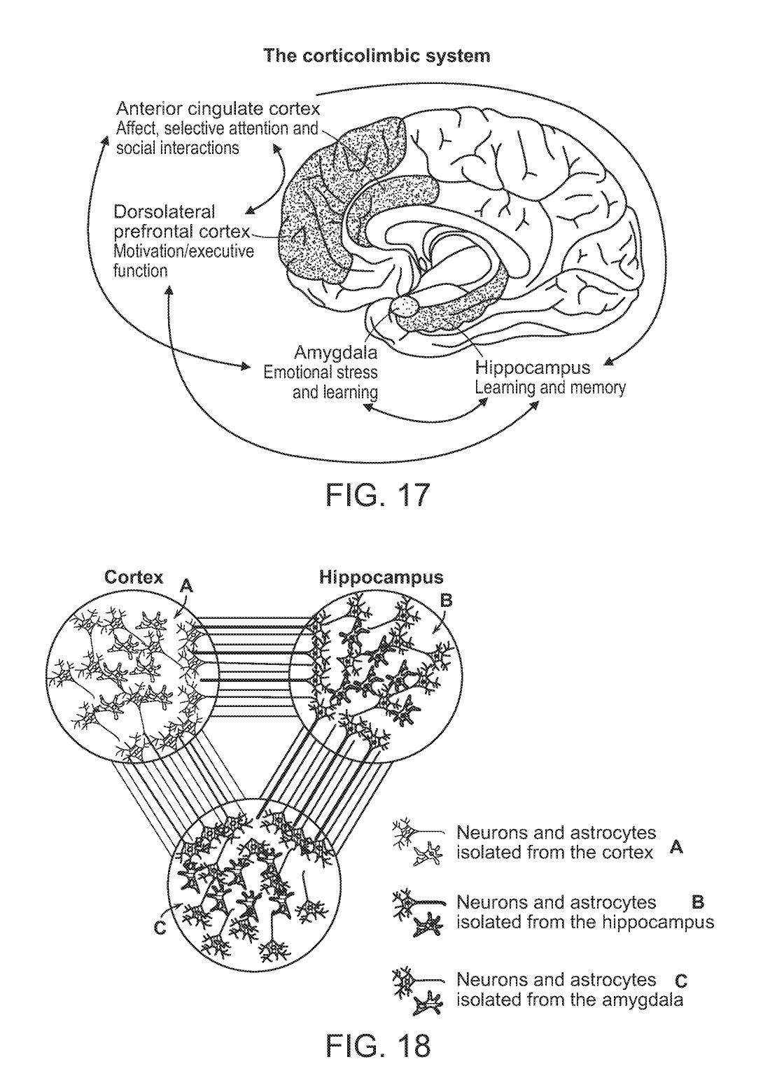

The exemplary in vitro trineural pathway model provides an electrically active neuronal circuit consisting of cells from three different brain regions, including the cortex, the hippocampus and the amygdala, which are connected via axons. FIG. 17 illustrates the corticolimbic system including the three different brain regions in the form of the cortex, the hippocampus and the amygdala. FIG. 18 illustrates functional interconnection of the cortex, hippocampus and amygdala neurons in the in vitro brain model. The in vitro trineural pathway model was fabricated using rodent primary brain cells, but, human neurons (either primary human neurons from different brain regions and/or iPSCs with brain region identity) can be used.

By implementing cells from the amygdala, the prefrontal cortex and the hippocampus, the trisynaptic pathway and part of the limbic lobe was rebuilt. Cells from different brain regions were seeded in separate compartments, which were separated by micro-printed lines (e.g., having dimensions of approximately 10×15 μm or 15×15 μm, representing the width of the line by the distance between the lines), which in turn guides the axons to functionally connect the compartments with each other. The complexity of essential neuronal connections was thereby added. The electrical activity (functionality), the ratio of various cell types, cell morphology and molecular and genetic markers for the specific brain regions can be defined.

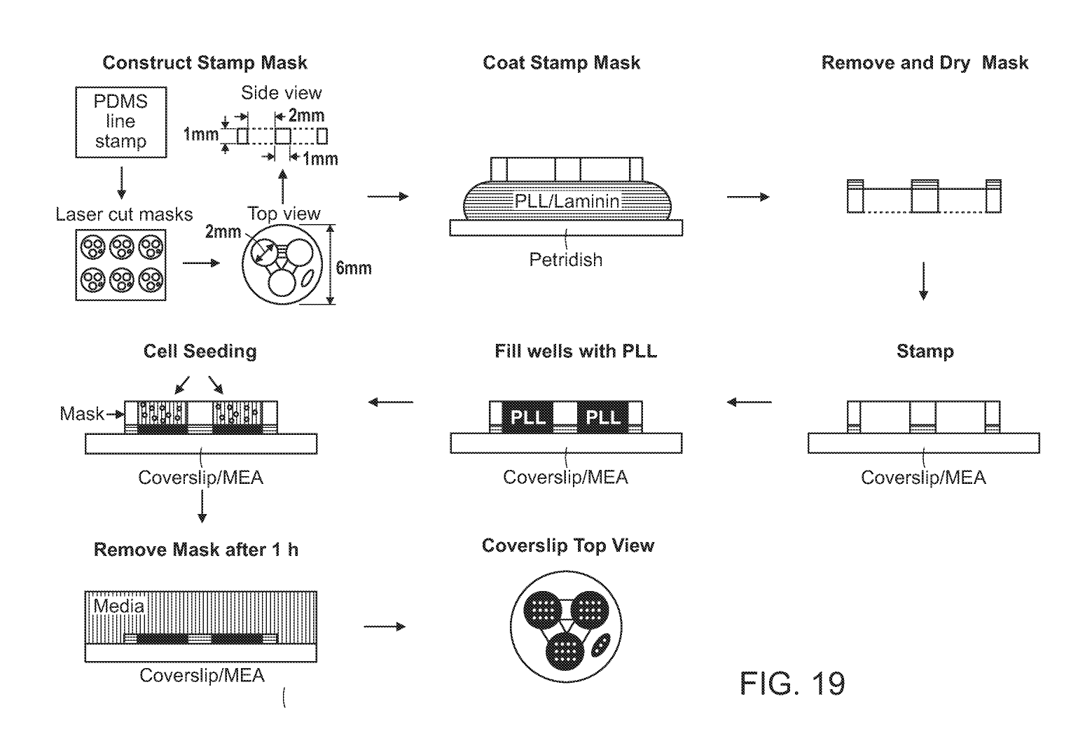

With reference to FIG. 19, a flowchart representing a process of forming the in vitro trineural pathway model is provided. In particular, the process of FIG. 19 uses microcontact printing to build a limbic lobe and schematically represents the microcontact printing process.

In order to separate the neuronal networks from each other and to control directionality of axonal growth, microcontact printing (See Quist, A. P. et al., Recent advances in microcontact printing, Analytical and Bioanalytical Chemistry, 381(3):591-600 (2005)) utilizing a polydimethylsiloxane (PDMS) mask in the shape as indicated in FIG. 18 was used. The masks were used for seeding the cells and were removed approximately one hour after seeding. After this amount of time, the cells attached to the substrate and stayed within their specific areas. In order to produce the PDMS mask, soft photolithography (See Betancourt, T. et al., Micro- and nanofabrication methods in nanotechnological medical and pharmaceutical devices, International Journal of Nanomedicine, 1(4):483-495 (2006)) was utilized. Specifically, a photomask with the desired line patterns (e.g., a set of thin lines) was created using computer-assisted design software and standard photolithography methods, and the mask was used to shadow a wafer glass covered with a photoresist when exposed to ultraviolet light. The thickness of the photoresist layer determines the depth of the grooves, and the width is determined by the photomask line patterns when the light-exposed areas of the photoresist are dissolved away. The etched surface was used as a “master” form onto which liquid PDMS silicon rubber was cast and allowed to cross-link. The product of this process is a PDMS stamp that can be used to either directly stamp (e.g., microcontact print, or the like) ECM molecules in patterns that match those designed into the master onto a culture substrate in desired patterns that similarly direct anisotropic tissue formation. The PDMS stamps were used to connect the round-shaped wells by thin lines, which provide the guidance for axonal growth to allow axons to cross the gap between the separate neuronal networks and connect them. Using the microcontact printing method, printing of desired ECM proteins in varying amount on the coverslip or the MEA is also possible. It should be understood that the shapes of the PDMS masks can be adjusted as desired. The cells for the in vitro model were isolated from the cortex, the hippocampus and the amygdala from two days old Sprague Dawley Rats. Cells were seeded as indicated in FIG. 19, either on coverslips or on a multi-electrode array (MEA), for morphological, genetic or functional readouts.

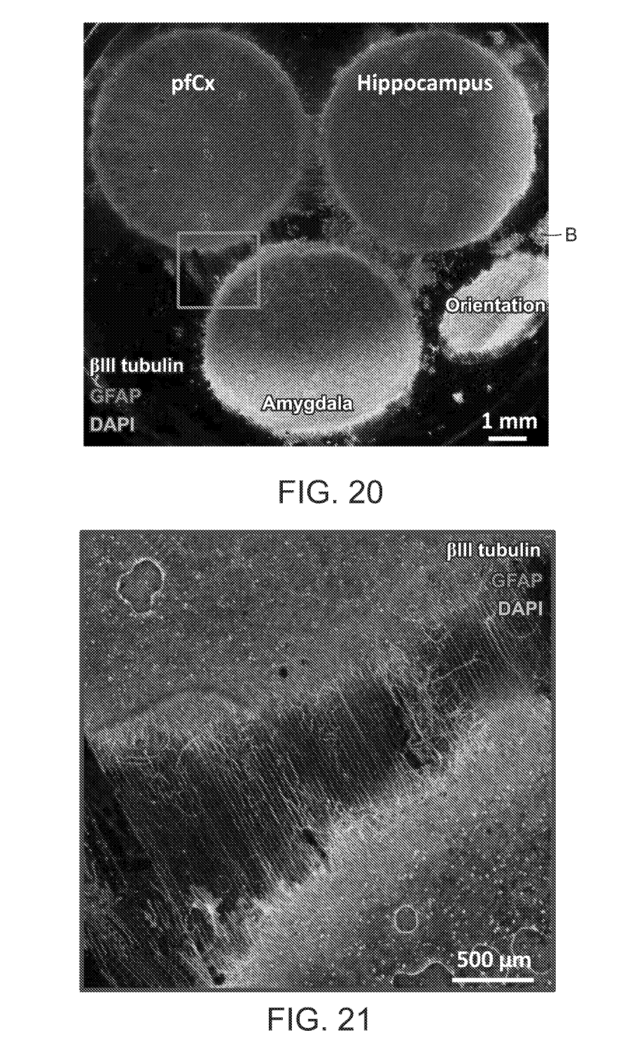

FIGS. 20 and 21 illustrate different magnifications of interconnected neuronal cells from the cortex, hippocampus and amygdala as generated during experimentation of an in vitro trineural pathway model. In particular, FIGS. 20 and 21 illustrate an immunostained trisynaptic pathway built by primary rat neurons from the prefrontal cortex (pfCx), the hippocampus and the amygdala (bIII tubulin represented by areas A and GFAP represented by areas B). The model of FIGS. 20 and 21 indicated a successful design and generation of an interconnected neuronal network of three different brain regions in vitro.

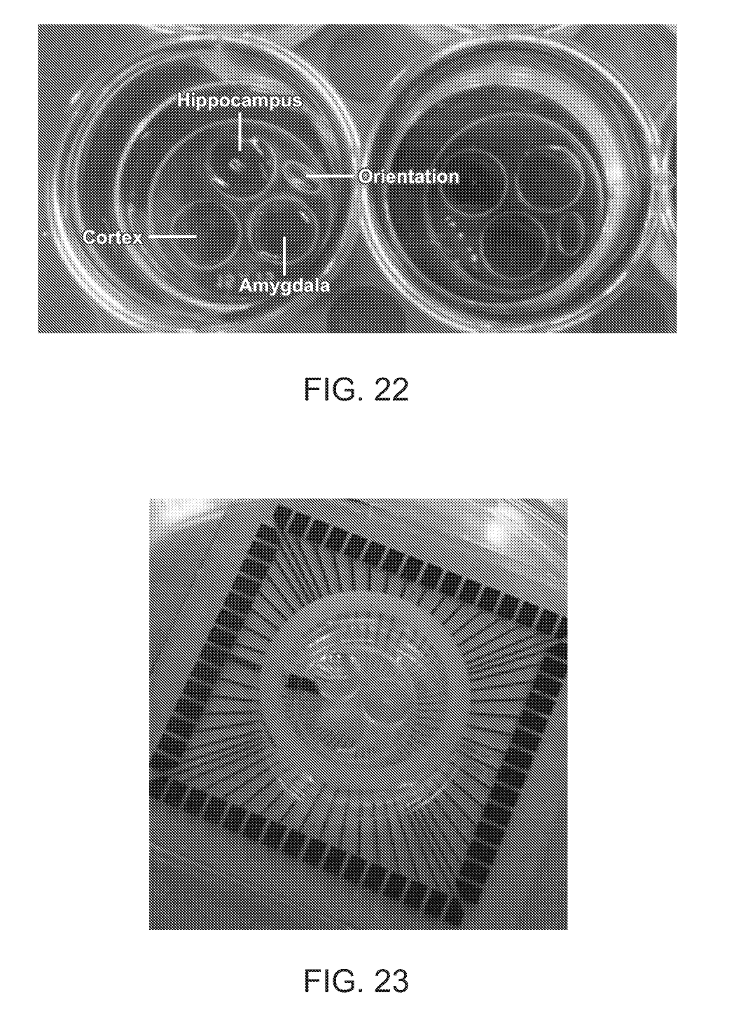

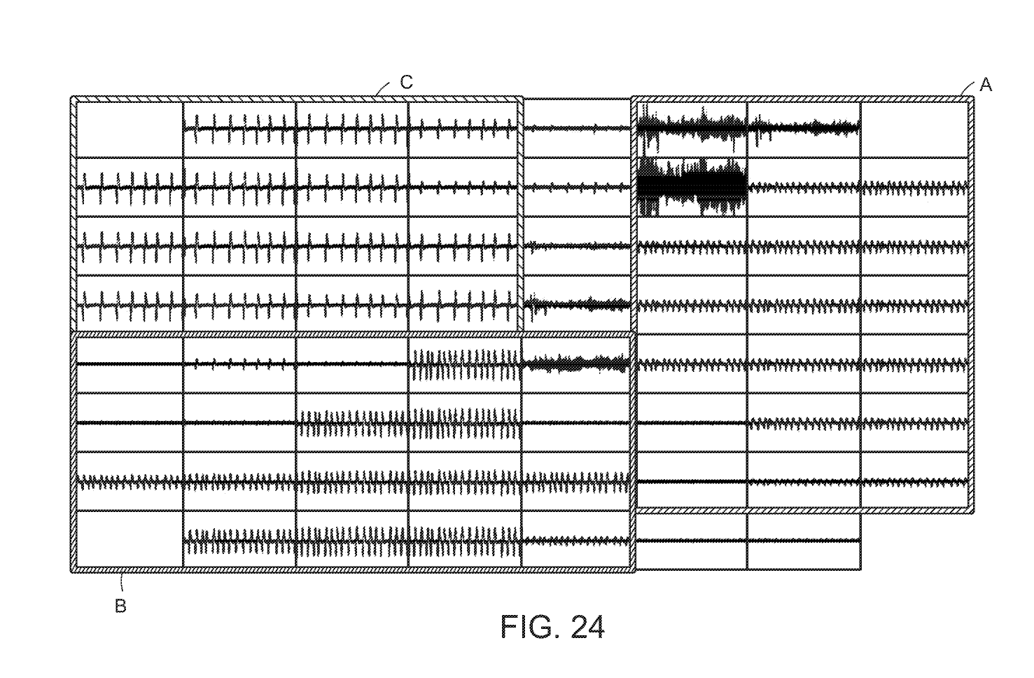

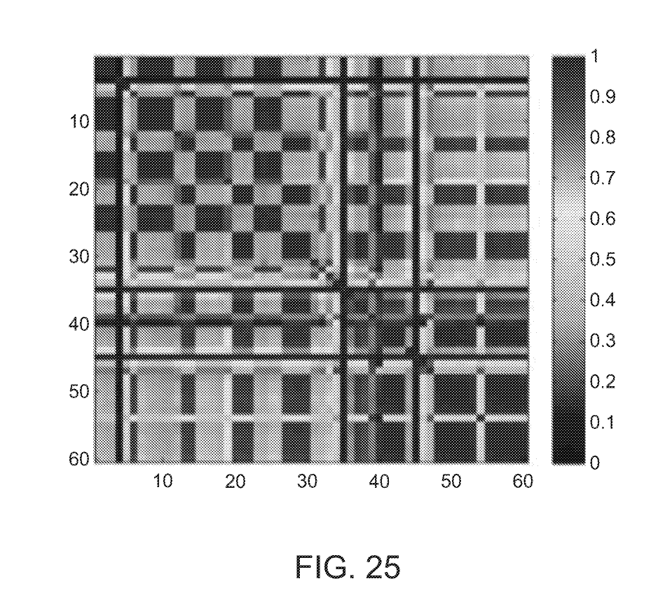

FIG. 22 illustrates an example image of the mask on a glass coverslip in a 12-well plate. FIG. 23 illustrates PDMS masks on a multi-electrode array (MEA). FIG. 24 illustrates electrical activity of cells from different brain regions in the in vitro trineural pathway model. Area A corresponds to the electrical activity of the prefrontal cortex, area B corresponds to the electrical activity of the hippocampus, and area C corresponds to the electrical activity of the amygdala. FIG. 25 illustrates a cross-correlation of electrical activity of the three different brain regions. In particular, FIG. 25 indicates that the different regions are communication with each other due to the functional an interconnected neuronal network achieved by the in vitro trineural pathway model.

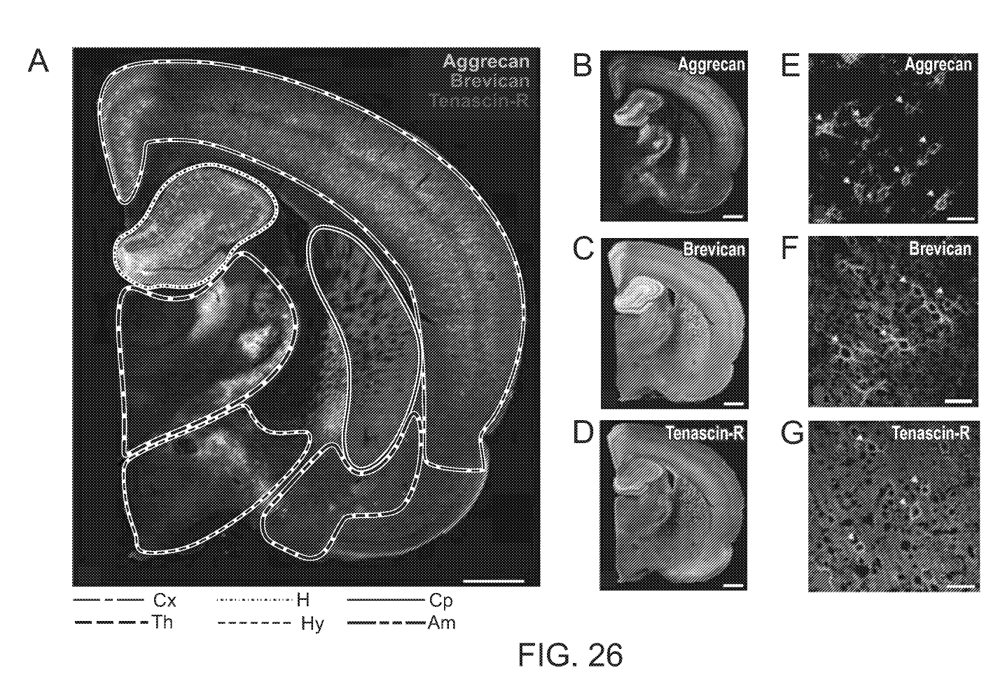

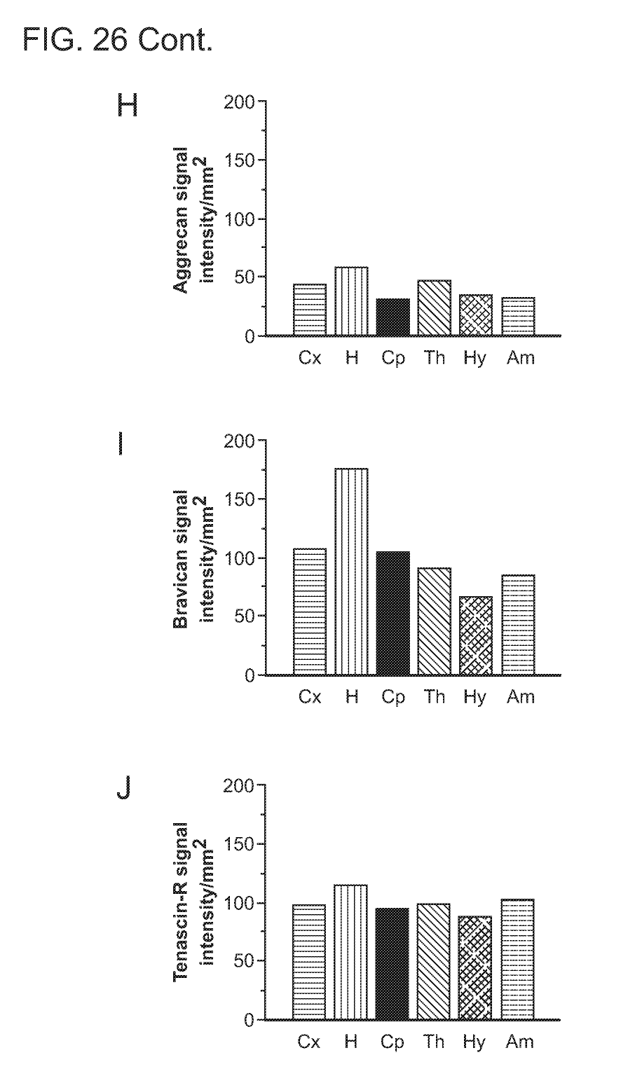

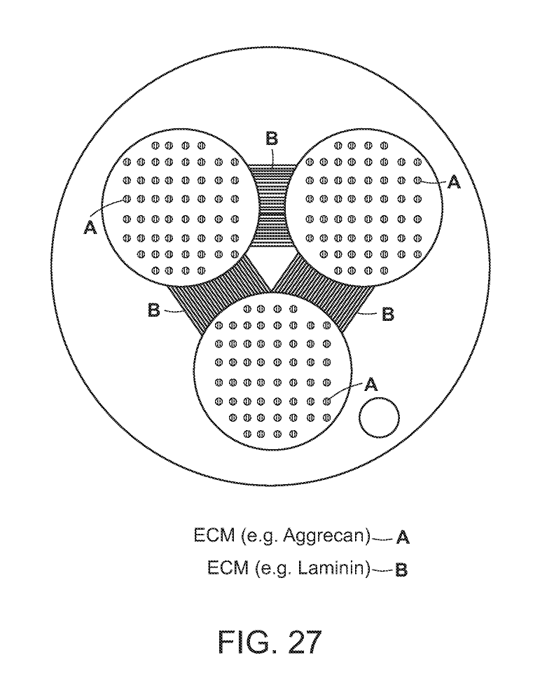

A detailed analysis was performed to determine in vivo and in vitro brain microenvironment characteristics of distinct brain regions for use in in vitro models described herein, including neurons isolated from the prefrontal cortex, hippocampus and amygdala. For example, FIGS. 26A-J illustrate examination of the extracellular matrix in vivo. In particular, FIGS. 26A-J provide an analysis of the distribution of brain extracellular matrix proteins, including Aggrecan, Brevican and Tenascin-R in order to implement the findings in the in vitro model. A mixture of ECM can be stamped onto a coverslip as depicted in FIG. 27. The mixture can be different for each brain region and can be adjusted according to the in vivo results. The coverslip of FIG. 27 illustrates a diagrammatic representation microcontact printed Aggrecan (and/or a mixture of ECM proteins) and Laminin (ECM) on a coverslip. Implementing distinct brain regions, their axonal connections and a brain-like microenvironment in the in vitro model advantageously provides an improved platform for accurately studying the brain and associated diseases or effects of toxicants.

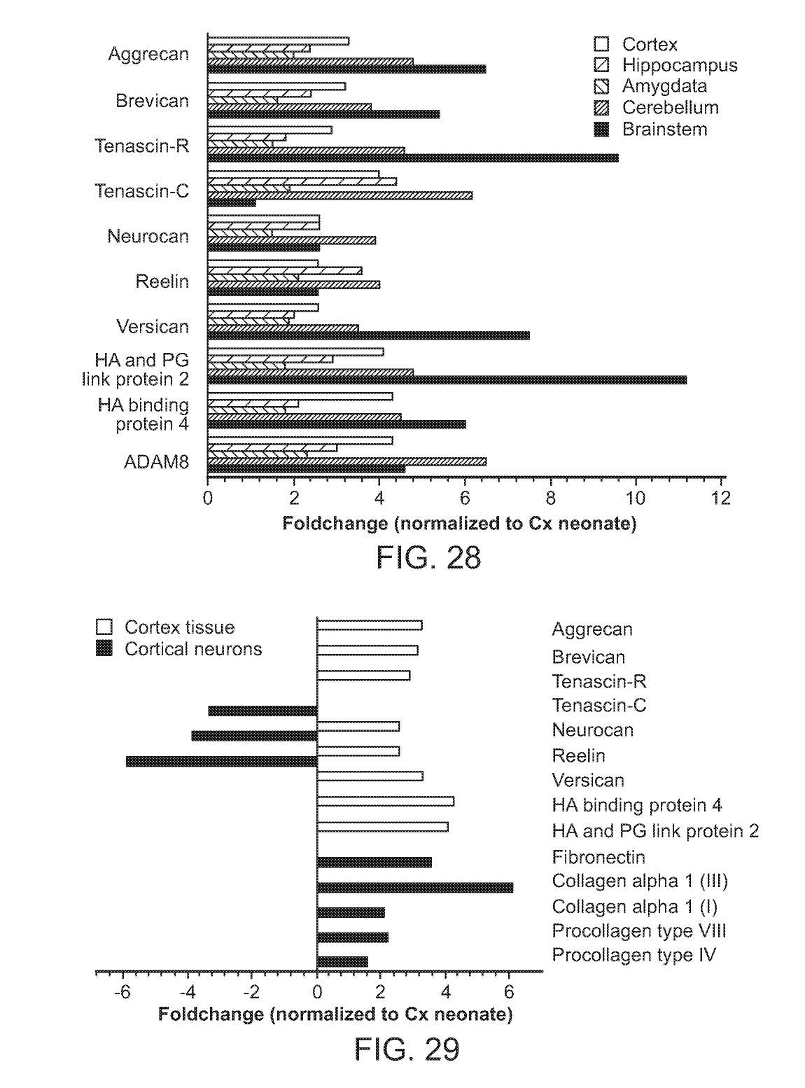

FIG. 28 illustrates ECM proteins in different regions of the brain. In particular, FIG. 28 illustrates that ECM proteins are highly expressed in the most exterior and exposed parts of the brain. FIG. 29 illustrates ECM protein expression as it differs in in vitro versus in vivo. It was determined that ECM protein distribution is brain region dependent and differs in vivo versus in vitro. Such findings were incorporated into the in vitro brain model to generate a more accurate model.

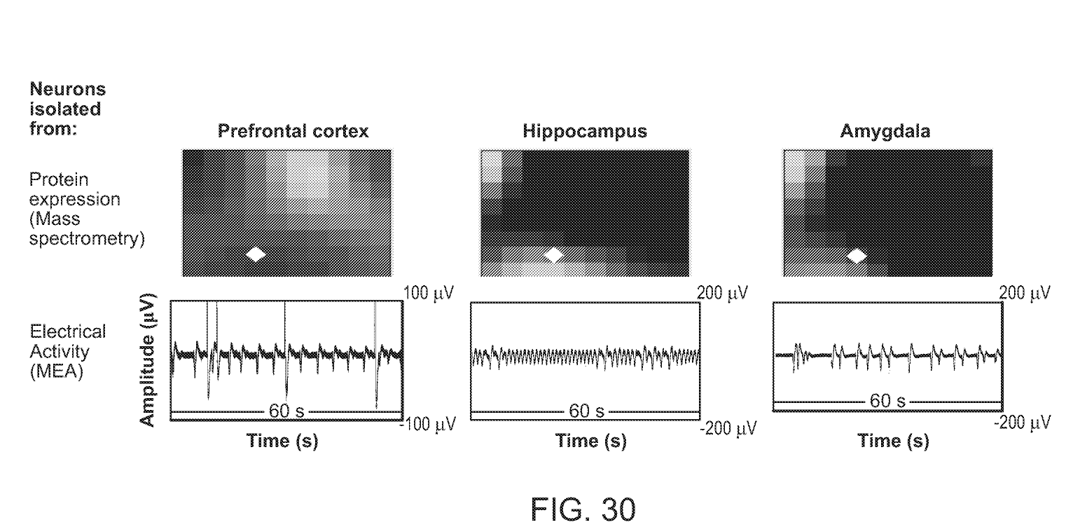

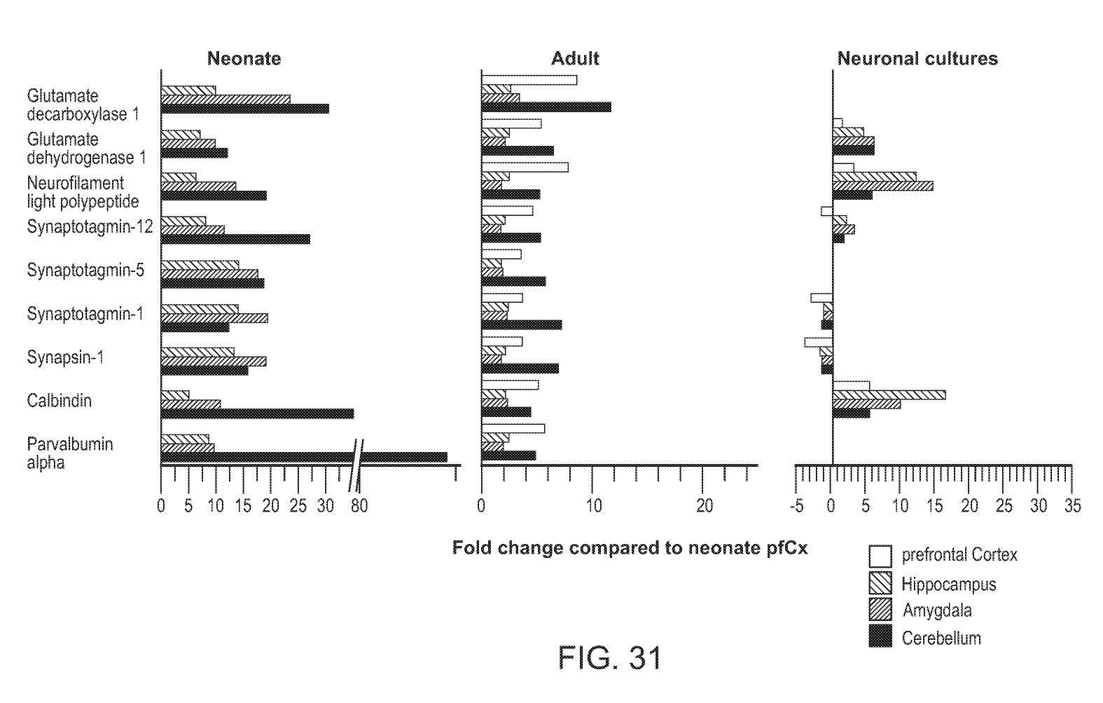

FIG. 30 illustrates the protein expression (mass spectrometry) and the electrical activity (MEA) for each of the isolated neurons. FIG. 31 illustrates a protein expression comparison of in vivo and in vitro neurons to determine how close the in vitro neurons mimic in vivo neurons based on fold change.

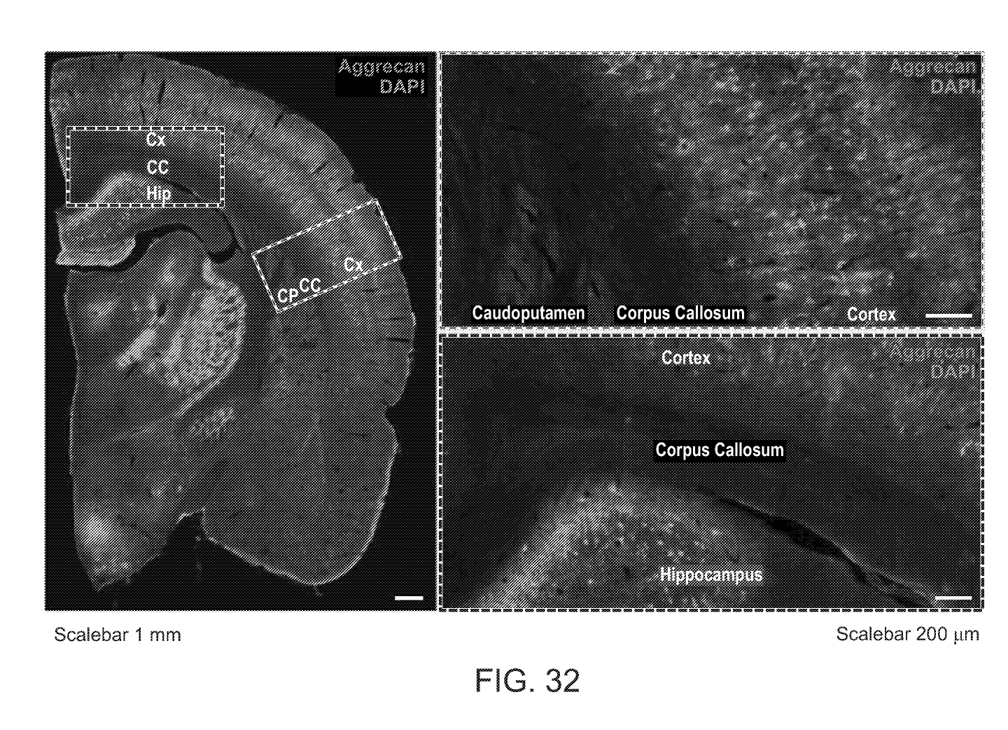

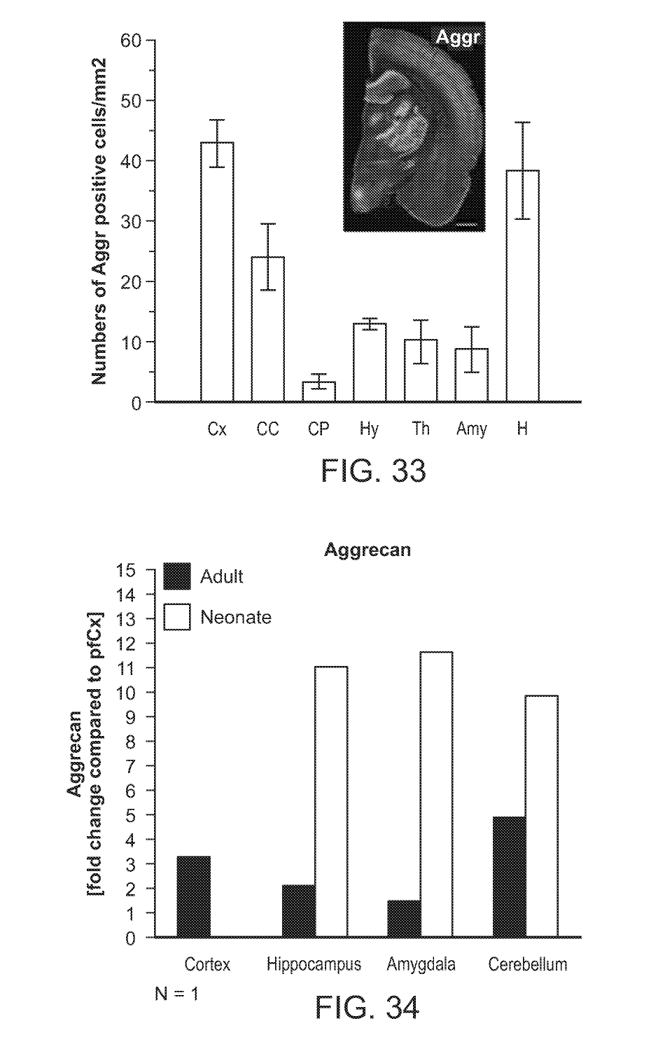

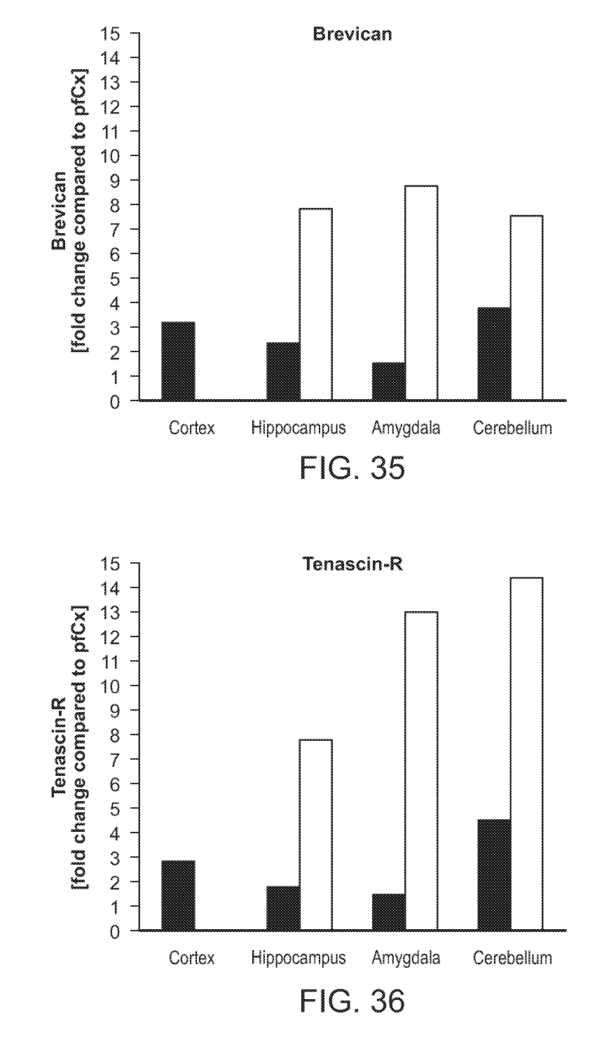

FIGS. 32-36 illustrate immunostaining and data relating to immunostaining of the microenvironment. ECM proteins were quantified throughout different rain regions and implemented in the in vitro model. Mass spectrometry was used to measure the number of Aggrecan positive cells, adult and neonate Aggrecan, Brevican and Tenascin-R. The ECM includes approximately 20% of the adult brain volume and is important for cell migration, neurogenesis, synaptic plasticity and stabilization, axonal path-finding, ionic buffering and neuroprotection. The brain has a unique ECM composition. Global and local distribution of the single ECM proteins was used.

The functionally connected trineural pathway models, devices, and systems of the present invention can be used in, for example, high throughput screening assays to determine the effects of a test compound on living tissue by examining the effect of the test compound on various biological responses, such as for example, a neural function, e.g., an electrophysiological function, cell viability, cell growth, migration, differentiation and maintenance of cell phenotype, metabolic activity, osmotic swelling, structural remodeling and tissue level pre-stress.

As used herein, the various forms of the term “modulate” are intended to include stimulation (e.g., increasing or upregulating a particular response or activity) and inhibition (e.g., decreasing or downregulating a particular response or activity).

As used herein, the term “contacting” (e.g., contacting an NVU or trineural pathway model with a test compound) is intended to include any form of interaction (e.g., direct or indirect interaction) of a test compound a brain model. The term contacting includes incubating a compound and a tissue or plurality of tissues together (e.g., adding the test compound to a tissue or plurality of a tissues in culture).

Test compounds, may be any agents including chemical agents (such as toxins), small molecules, pharmaceuticals, peptides, proteins (such as antibodies, cytokines, enzymes, and the like), nanoparticles, and nucleic acids, including gene medicines and introduced genes, which may encode therapeutic agents, such as proteins, antisense agents (i.e., nucleic acids comprising a sequence complementary to a target RNA expressed in a target cell type, such as RNAi or siRNA), ribozymes, and the like.

The test compound may be added to a tissue by any suitable means. For example, the test compound may be added drop-wise onto the surface of a device of the invention and allowed to diffuse into or otherwise enter the device, or it can be added to the nutrient medium and allowed to diffuse through the medium. In the embodiment where the device of the invention comprises a multi-well plate, each of the culture wells may be contacted with a different test compound or the same test compound. In one embodiment, the screening platform includes a microfluidics handling system to deliver a test compound and simulate exposure of the microvasculature to drug delivery. In one embodiment, a solution comprising the test compound may also comprise fluorescent particles, and a cell function may be monitored using Particle Image Velocimetry (PIV).

Numerous physiologically relevant parameters, e.g., neural cell activities, can be evaluated using the methods and devices of the invention. For example, in one embodiment, the devices of the present invention can be used in assays which measure a neural function, such as neuronal firing frequency and/or amplitude.

In yet another embodiment, the devices of the present invention can be used in pharmacological assays for measuring the effect of a test compound on the stress state of a tissue. For example, the assays may involve determining the effect of a drug on tissue stress and structural remodeling of the tissue.

In further embodiments, the devices of the present invention can be used to study functional differentiation of stem cells (e.g., pluripotent stem cells, multipotent stem cells, induced pluripotent stem cells, and progenitor cells of embryonic, fetal, neonatal, juvenile and adult origin) into neuronal phenotypes.

### EQUIVALENTS

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements or method steps, those elements or steps may be replaced with a single element or step. Likewise, a single element or step may be replaced with a plurality of elements or steps that serve the same purpose. Further, where parameters for various properties are specified herein for exemplary embodiments, those parameters may be adjusted up or down by 1/20th, 1/10th, ⅕th, ⅓rd, ½, etc., or by rounded-off approximations thereof, unless otherwise specified. Moreover, while exemplary embodiments have been shown and described with references to particular embodiments thereof, those of ordinary skill in the art will understand that various substitutions and alterations in form and details may be made therein without departing from the scope of the invention. Further still, other aspects, functions and advantages are also within the scope of the invention.

Exemplary flowcharts are provided herein for illustrative purposes and are non-limiting examples of methods. One of ordinary skill in the art will recognize that exemplary methods may include more or fewer steps than those illustrated in the exemplary flowcharts, and that the steps in the exemplary flowcharts may be performed in a different order than shown.

### INCORPORATION BY REFERENCE

The contents of all references, including patents and patent applications, cited throughout this application are hereby incorporated herein by reference in their entirety. The appropriate components and methods of those references may be selected for the invention and embodiments thereof. Still further, the components and methods identified in the Background section are integral to this disclosure and can be used in conjunction with or substituted for components and methods described elsewhere in the disclosure within the scope of the invention.

1. A structure for growth and development or support of an engineered neurovascular unit (NVU), the NVU structure comprising:
a hyaluronic acid scaffold configured to support neuron and astrocyte growth and direct development of neuronal tissue;
a first channel within the hyaluronic acid scaffold configured to support endothelial and pericyte cell growth; and
a second channel within the hyaluronic acid scaffold and parallel to the first channel configured to support flow of angiogenic factors and the formation of gradients perpendicular to the first channel and direct the formation of capillary-like channels from the first channel.

2. The NVU structure of claim 1, further comprising a channel input and a channel output connected to opposing sides of the first channel; or a channel input and a channel output connected to opposing sides of the second channel.

3. (canceled)

4. The NVU structure of claim 1, wherein the angiogenic factors are at least one of VEGF, b-FGF, or PMA.

5. The NVU structure of claim 1, wherein the NVU structure is configured to promote formation of capillary-like channels endothelial cells, the capillary-like structures extending from the first channel and mimicking in vivo cell formation.

6. A device for in vitro evaluation of a neurovascular unit (NVU), comprising:
the NVU structure of claim 1;
and
a housing including:
a chamber configured to house the NVU structure;
an input channel connected to the chamber configured to house the engineered NVU; and
an output channel connected to the chamber configured to house the engineered NVU.

7. A system for in vitro evaluation of a neurovascular unit (NVU), the system comprising:
a plurality of the NVU structures of claim 1,
and
a housing including:
a plurality of chambers, each chamber configured to independently house at least one of the NVU structures;
a plurality of input channels, each input channel independently connected to at least one of the plurality of chambers; and
a plurality of output channels, each output channel independently connected to at least one of the plurality of chambers.

8. A method for identifying a compound which modulates an NVU function, comprising:
providing the device of claim 6, the device further comprising neuronal cells, astrocytes, endothelial cells, and pericytes forming an NVU unit;
contacting the NVU with a test compound; and
determining the effect of the test compound on an NVU function in the presence and absence of the test compound, wherein a modulation of the NVU function in the presence of said test compound as compared to the NVU function in the absence of said test compound indicates that said test compound modulates an NVU function, thereby identifying a compound that modulates an NVU function.

9. The method of claim 8, wherein the NVU function is selected from the group consisting of neuronal firing frequency, neuronal firing amplitude, capillary diameter, and vasomotor tone.

10.-12. (canceled)

13. An engineered functionally connected trineural pathway, comprising:
a base layer including a first region, a second region, and a third region, wherein the first region, the second region, and the third region are spatially separated from each other;
a first independent plurality of cells comprising neurons and astrocytes obtained from a first brain region, the first independent plurality of cells adhered to the first region of the base layer;
a second independent plurality of cells comprising neurons and astrocytes obtained from a second brain region, the second independent plurality of cells adhered to the second region of the base layer;
a third independent plurality of cells comprising neurons and astrocytes obtained from a third brain region, the third independent plurality of cells adhered to the third region of the base layer;
a first independent plurality of axons adhered to the base layer and functionally connecting the first plurality of cells to the second plurality of cells;
a second independent plurality of axons adhered to the base layer and functionally connecting the first plurality of cells to the third plurality of cells; and
a third independent plurality of axons adhered to the base layer and functionally connecting the second plurality of cells to the third plurality of cells.

14. The engineered functionally connected trineural pathway of claim 13, wherein the first brain region is the cortex.

15. The engineered functionally connected trineural pathway of claim 13, wherein the second brain region is the hippocampus.

16. The engineered functionally connected trineural pathway of claim 13, wherein the third brain region is the amygdala.

17. A device for in vitro evaluation of a neural function and/or a neural morphology, comprising:
the engineered functionally connected trineural pathway of claim 13,
and
a housing including:
a chamber configured to house the engineered functionally connected trineural pathway;
an input channel connected to the chamber housing the engineered functionally connected trineural pathway; and
an output channel connected to the chamber housing the engineered functionally connected trineural pathway.

18. A system for in vitro evaluation of a neural function and/or a neural morphology, the system comprising:
a plurality of the engineered functionally connected trineural pathways of claim 13,
and
a housing including:
a plurality of chambers, each chamber configured to independently house at least one of the plurality of engineered functionally connected trineural pathways;
a plurality of input channels, each input channel independently connected to at least one of the plurality of chambers; and
a plurality of output channels, each output channel independently connected to at least one of the plurality of chambers.

19. A method for identifying a compound which modulates a neural function and/or a neural morphology, comprising:
providing the device of claim 17;
contacting the functionally connected trineural pathway with a test compound; and
determining the effect of the test compound on a neural function and/or a neural morphology in the presence and absence of the test compound, wherein a modulation of the neural function and/or neural morphology in the presence of said test compound as compared to the neural function and/or neural morphology in the absence of said test compound indicates that said test compound modulates a neural function and/or a neural morphology, thereby identifying a compound that modulates a neural function and/or a neural morphology.

20. The method of claim 19, wherein the neural function is an electrical function.

21. A method of forming an engineered functionally connected trineural pathway, comprising:
providing a base layer including a first region, a second region, and a third region, wherein the first region, the second region, and the third region are spatially separated from each other;
depositing a pattern of extracellular matrix protein onto the base layer, wherein the pattern comprises a first plurality of lines connecting the first region with the second region, a second plurality of lines connecting the first region with the third region, and a third plurality of lines connecting the second region with the third region,
placing a patterned mask onto the base layer, wherein the patterned mask includes a first opening, a second opening, and a third opening corresponding to the first, second and third regions, respectively, seeding neural cells and astrocytes obtained from a first area of the brain onto the first region through the first opening; seeding neural cells and astrocytes obtained from a second area of the brain onto the second region through the second opening; and seeding neural cells and astrocytes obtained from a third area of the brain onto the third region through the third opening;
culturing the cells for a time sufficient for the cells in each region to adhere to the base layer;
removing the patterned mask and further culturing the cells for a time sufficient for the cells from the first and second areas of the brain to functionally connect, for the cells from the first and third areas of the brain to functionally connect, and for the cells from the second and third areas of the brain to functionally connect, thereby generating the engineered functionally connected trineural pathway.

22. The method of claim 21, wherein the base layer is a glass coverslip or a petri dish.

23. The method of claim 21, wherein the extracellular matrix protein is selected from the group consisting of fibronectin, laminin, bevican, aggrecan, tenascin-R, and combinations thereof.

24. The method of claim 21, wherein the neural cells and astrocytes are independently obtained from a cortex region, a hippocampus region, and an amygdala region of the brain.

25. The method of claim 21, comprising depositing a cell adherent substance on one or more of the three regions on the base layer.

26. The method of claim 25, wherein the cell adherent substance is poly-L-lysine.

27. The device of claim 6, wherein the channel input and the channel are connected to opposing sides of the first channel; or wherein the channel input and the channel output are connected to opposing sides of the second channel.
